(19) United States
(12) Patent Application Publication
(10) Pub. No.: US 2022/0399126 A1
(43) Pub. Date: Dec. 15, 2022

(54) COMPUTER SYSTEMS AND METHODS FOR MACHINE-LEARNING BASED TREATMENT MODELING FOR ONCOLOGY BASED ON INCONSISTENT RAS BIOMARKER DETECTION DATA RECORDS

(71) Applicant: Optum, Inc., Minnetonka, MN (US)

(72) Inventors: Christine M. John, Hermitage, TN (US); Lorre Ann Ochs, Eden Prairie, MN (US); Lynn Anne Richards, Brooklyn Park, MN (US); Fang Guo, Apex, NC (US); Liping Fan, Basking Ridge, NJ (US); Julia Vaynerman, Eagan, MN (US)

(21) Appl. No.: 17/344,510

(22) Filed: Jun. 10, 2021

(51) Int. Cl. G16H 50/50; G06N 20/00; G06F 30/27

(52) U.S. Cl. CPC G16H 50/50; G06N 20/00; G06F 30/27

(57) ABSTRACT

To enable automated processing of certain observation data by machine-learning based models, as well as to ensure those machine-learning based models are trained utilizing reliable and consistent data, independently generated observation data records each comprising biomarker mutation indicators, are utilized to generate a model input data set by applying biomarker mutation indicator-based filters, including intra-date filters and inter-date filters to identify and rectify observation data records that do not satisfy applicable biomarker mutation indicators. By identifying and rectifying the observation data records that do not satisfy applicable biomarker mutation indicators, a clean, model input data set is generated that is then utilized to generate a severity score for the patient.

### BACKGROUND

Oncological diagnosis and treatment of many cancer types often results in a large number of data records for each patient. Those data records generally encompass clinical data records (e.g., including indications of one or more diagnoses associated with the patient, memorializing notes of a medical care provider, lab data records indicating the results of lab tests performed on the patient, and/or the like), claims data records encompassing data submitted to a medical care payor for the patient, and/or the like. These data records are generated independently from one another, which can periodically result in inconsistent data describing aspects of a patient's cancer and/or treatment. These inconsistencies within data for a particular patient may inhibit use of automated computer-based models that rely on consistent data to produce accurate model outputs.

Accordingly, a need exists for systems and methods that enable computer-implemented models to intake and reconcile potentially inconsistent data sets for use with computer-implemented data models.

### BRIEF SUMMARY

Embodiments as discussed herein are configured to intake a plurality of independently generated, discrete observation data records received from a defined data source, and to prepare the received set of observation data records to be utilized as input for other analytics, such as for ingestion by a machine-learning severity model. The plurality of observation data records that encompass the received set of observation data records comprise observation data elements relevant to a particular patient, and each observation data record comprises data identifying a relevant date of the observation data record and data indicative of specific biomarker mutations encompassing RAS (or more specific mutations of RAS, including KRAS and NRAS) detected within cancer cells of the patient.

Certain embodiments are directed to a computer-implemented method for automatically modeling severity attributes of a colon cancer or rectal cancer treatment utilizing a plurality of independently generated observation data records for a patient. In certain embodiments, the method comprises: receiving a plurality of independently generated observation data records each comprising a biomarker mutation indicator relating to RAS, KRAS, or NRAS and wherein KRAS and NRAS are subsets of RAS; generating a model input data set comprising a subset of the plurality of independently generated observation data records at least in part by: applying a plurality of biomarker mutation indicator-based filters comprising: an intra-date filter configured to identify a plurality of observation data records having a shared date-of-service and to eliminate one or more observation data records failing to satisfy the intra-date filter; and an inter-date filter configured to identify a plurality of observation data records across a plurality of dates of service and to eliminate one or more observation data records failing to satisfy the inter-date filter; wherein each of the plurality of biomarker mutation indicator-based filters are configured to identify observation data records failing to satisfy the biomarker mutation indicator-based filters across RAS, KRAS, and NRAS biomarker mutation indicators; after applying the plurality of biomarker mutation indicator-based filters to identify the subset of the plurality of independently generated observation data records, generating a derived biomarker mutation indicator for each of the subset of the plurality of independently generated observation data records, wherein the derived biomarker mutation indicator is a derived RAS biomarker mutation indicator generated based at least in part on the biomarker mutation indicator included within a respective observation data record; providing the model input data set to a machine-learning severity model configured to generate severity data based at least in part on the derived RAS biomarker mutation indicators of the observation data records within the model input data set; and generating, via the machine-learning severity model, severity data relating to the patient.

In certain embodiments, generating a model input data set comprises generating a flat model input data file comprising each of the subset of the plurality of observation data records. In various embodiments, the intra-date filter is configured to identify a first observation data record having the shared date-of-service and having a positive result indicator of at least one of RAS, KRAS, or NRAS and a second observation data record having the shared date-of-service and having a negative result indicator of at least one of RAS, KRAS, or NRAS and to eliminate the first observation data record and the second observation data record as failing to satisfy the intra-date filter. In certain embodiments, the intra-date filter is configured to identify a first observation data record having the shared date-of-service and having a positive result indicator of RAS, a second observation data record having the shared date-of-service and having a negative result indicator of KRAS, and a third observation data record having the shared date-of-service and having a negative result indicator of NRAS and to eliminate the first observation data record, the second observation data record, and the third observation data record as failing to satisfy the intra-date filter. In certain embodiments, the intra-date filter is configured to identify a first clinical record having the shared date-of-service and having a negative result indicator of RAS and a second observation data record having the shared-date-of-service and having a positive result indicator of at least one of KRAS or NRAS and to eliminate the first observation data record and the second observation data record as failing to satisfy the intra-date filter. In various embodiments, the inter-date filter is configured to identify a first observation data record having a first date-of-service and having a positive result indicator of at least one RAS, KRAS, or NRAS and a second observation data record having a second date-of-service occurring after the first date-of-service and having a negative result indicator of at least one of RAS, KRAS, or NRAS and to eliminate the first observation data record and the second observation data record as failing to satisfy the inter-date filter. Moreover, the inter-date filter may be configured to identify a first observation data record having a first date-of-service having a positive result indicator of at least one NRAS or KRAS and a second observation data record having a second date-of-service occurring after the first-date-of-service and having a negative result indicator of RAS and to eliminate the first observation data record and the second observation data record as failing to satisfy the inter-date filter. In certain embodiments, the inter-date filter is configured to identify a first observation data record having a first date-of-service and having a positive result indicator of at least one of RAS, KRAS, or NRAS and having a second observation data record having a second date-of-service occurring after the first date-of-service and having a negative result indicator of at least one of KRAS or NRAS and to eliminate the second observation data record as failing to satisfy the output filter. In various embodiments, generating a model input data set is performed in accordance with a relevant data pre-processing methodology relating to colon cancer or rectal cancer, and wherein the method further comprises retrieving the relevant data pre-processing methodology from a plurality of data pre-processing methodologies based at least in part on the plurality of independently generated observation data records prior to generating the model input data set. In certain embodiments, the intra-date filter is configured to eliminate one or more observation data records failing to satisfy the intra-date filter relating to one of RAS, KRAS, or NRAS; and the inter-date filter is configured to eliminate one or more observation data records failing to satisfy the inter-date filter relating to one of RAS, KRAS, or NRAS. In certain embodiments, the method further comprises applying a preliminary filter criteria before generating the model input data set, wherein the preliminary filter criteria comprise one or more of: a date-based filter criterion for selecting independently generated observation data records for further analysis as generated within a defined date range; a data source filter criterion for selecting independently generated observation data records for further analysis as generated by one or more defined data sources; or a data content filter criterion for selecting independently generated observation data records for further analysis as containing an identifier selected from a plurality of available identifiers eligible for further analysis. In various embodiments, the machine-learning severity model is a linear regression model.

Certain embodiments are directed to a system comprising one or more memory storage areas and one or more processors for automatically modeling severity attributes of a colon cancer or rectal cancer treatment utilizing a plurality of independently generated observation data records for a patient, the one or more processors are collectively configured to: receive a plurality of independently generated observation data records each comprising a biomarker mutation indicator relating to RAS, KRAS, or NRAS and wherein KRAS and NRAS are subsets of RAS; generate a model input data set comprising a subset of the plurality of independently generated observation data records at least in part by: apply a plurality of biomarker mutation indicator-based filters comprising: an intra-date filter configured to identify a plurality of observation data records having a shared date-of-service and to eliminate one or more observation data records failing to satisfy the intra-date filter; and an inter-date filter configured to identify a plurality of observation data records across a plurality of dates of service and to eliminate one or more observation data records failing to satisfy the inter-date filter; wherein each of the plurality of biomarker mutation indicator-based filters are configured to identify observation data records failing to satisfy the biomarker mutation indicator-based filters across RAS, KRAS, and NRAS biomarker mutation indicators; after applying the plurality of biomarker mutation indicator-based filters to identify the subset of the plurality of independently generated observation data records, generate a derived biomarker mutation indicator for each of the subset of the plurality of independently generated observation data records, wherein the derived biomarker mutation indicator is a derived RAS biomarker mutation indicator generated based at least in part on the biomarker mutation indicator included within a respective observation data record; provide the model input data set to a machine-learning severity model configured to generate severity data based at least in part on the derived RAS biomarker mutation indicators of the observation data records within the model input data set; and generate, via the machine-learning severity model, severity data relating to the patient.

In various embodiments, generating a model input data set comprises generating a flat model input data file comprising each of the subset of the plurality of observation data records. Moreover, the intra-date filter may be configured to identify a first observation data record having the shared date-of-service and having a positive result indicator of at least one of RAS, KRAS, or NRAS and a second observation data record having a shared biomarker mutation indicator and the shared date-of-service and having a negative result indicator of at least one of RAS, KRAS, or NRAS and to eliminate the first observation data record and the second observation data record as failing to satisfy the intra-date filter. In certain embodiments, the intra-date filter is configured to identify a first observation data record having the shared date-of-service and having a positive result indicator of RAS, a second observation data record having the shared date-of-service and having a negative result indicator of KRAS, and a third observation data record having the shared date-of-service and having a negative result indicator of NRAS and to eliminate the first observation data record, the second observation data record, and the third observation data record as failing to satisfy the intra-date filter. In various embodiments, the intra-date filter is configured to identify a first clinical record having the shared date-of-service and having a negative result indicator of RAS and a second observation data record having the shared-date-of-service and having a positive result indicator of at least one of KRAS or NRAS and to eliminate the first observation data record and the second observation data record as failing to satisfy the intra-date filter. In certain embodiments, the inter-date filter is configured to identify a first observation data record having a first date-of-service and having a positive result indicator of at least one RAS, KRAS, or NRAS and a second observation data record having a second date-of-service occurring after the first date-of-service and having a negative result indicator of at least one of RAS, KRAS, or NRAS and to eliminate the first observation data record and the second observation data record as failing to satisfy the inter-date filter. Moreover, the inter-date filter may be configured to identify a first observation data record having a first date-of-service having a positive result indicator of at least one NRAS or KRAS and a second observation data record having a second date-of-service occurring after the first-date-of-service and having a negative result indicator of RAS and to eliminate the first observation data record and the second observation data record as failing to satisfy the inter-date filter. In certain embodiments, the inter-date filter is configured to identify a first observation data record having a first date-of-service and having a positive result indicator of at least one of RAS, KRAS, or NRAS and having a second observation data record having a second date-of-service occurring after the first date-of-service and having a negative result indicator of at least one of KRAS or NRAS and to eliminate the second observation data record as failing to satisfy the output filter. Moreover, generating a model input data set is performed in accordance with a relevant data pre-processing methodology relating to colon cancer or rectal cancer, and wherein the method further comprises retrieving the relevant data pre-processing methodology from a plurality of data pre-processing methodologies based at least in part on the plurality of independently generated observation data records prior to generating the model input data set. In certain embodiments, the intra-date filter is configured to eliminate one or more observation data records failing to satisfy the intra-date filter relating to one of RAS, KRAS, or NRAS; and the inter-date filter is configured to eliminate one or more observation data records failing to satisfy the inter-date filter relating to one of RAS, KRAS, or NRAS.

In various embodiments, the one or more processors are additionally configured to apply a preliminary filter criteria before generating the model input data set, wherein the preliminary filter criteria comprise one or more of: a date-based filter criterion for selecting independently generated observation data records for further analysis as generated within a defined date range; a data source filter criterion for selecting independently generated observation data records for further analysis as generated by one or more defined data sources; or a data content filter criterion for selecting independently generated observation data records for further analysis as containing an identifier selected from a plurality of available identifiers eligible for further analysis. Moreover, the machine-learning severity model may be a linear regression model.

Various embodiments are directed to a computer program product for automatically modeling severity attributes of a colon cancer or rectal cancer treatment utilizing a plurality of independently generated observation data records for a patient, the computer program product comprising at least one non-transitory computer-readable storage medium having computer-readable program code portions stored therein, the computer-readable program code portions configured to: receive a plurality of independently generated observation data records each comprising a biomarker mutation indicator relating to RAS, KRAS, or NRAS and wherein KRAS and NRAS are subsets of RAS; generate a model input data set comprising a subset of the plurality of independently generated observation data records at least in part by: apply a plurality of biomarker mutation indicator-based filters comprising: an intra-date filter configured to identify a plurality of observation data records having a shared date-of-service and to eliminate one or more observation data records failing to satisfy the intra-date filter; and an inter-date filter configured to identify a plurality of observation data records across a plurality of dates of service and to eliminate one or more observation data records failing to satisfy the inter-date filter; wherein each of the plurality of biomarker mutation indicator-based filters are configured to identify observation data records failing to satisfy the biomarker mutation indicator-based filters across RAS, KRAS, and NRAS biomarker mutation indicators; after applying the plurality of biomarker mutation indicator-based filters to identify the subset of the plurality of independently generated observation data records, generate a derived biomarker mutation indicator for each of the subset of the plurality of independently generated observation data records, wherein the derived biomarker mutation indicator is a RAS biomarker mutation indicator generated based at least in part on the biomarker mutation indicator included within a respective observation data record; provide the model input data set to a machine-learning severity model configured to generate severity data based at least in part on the derived RAS biomarker mutation indicators of the observation data records within the model input data set; and generate, via the machine-learning severity model, severity data relating to the patient.

In certain embodiments, generating a model input data set comprises generating a flat model input data file comprising each of the subset of the plurality of observation data records. Moreover, the intra-date filter may be configured to identify a first observation data record having the shared date-of-service and having a positive result indicator of at least one of RAS, KRAS, or NRAS and a second observation data record having the shared date-of-service and having a negative result indicator of at least one of RAS, KRAS, or NRAS and to eliminate the first observation data record and the second observation data record as failing to satisfy the intra-date filter. In certain embodiments, the intra-date filter is configured to identify a first observation data record having the shared date-of-service and having a positive result indicator of RAS, a second observation data record having the shared date-of-service and having a negative result indicator of KRAS, and a third observation data record having the shared date-of-service and having a negative result indicator of NRAS and to eliminate the first observation data record, the second observation data record, and the third observation data record as failing to satisfy the intra-date filter. In various embodiments, the intra-date filter is configured to identify a first clinical record having the shared date-of-service and having a negative result indicator of RAS and a second observation data record having the shared-date-of-service and having a positive result indicator of at least one of KRAS or NRAS and to eliminate the first observation data record and the second observation data record as failing to satisfy the intra-date filter. In various embodiments, the inter-date filter is configured to identify a first observation data record having a first date-of-service and having a positive result indicator of at least one RAS, KRAS, or NRAS and a second observation data record having a second date-of-service occurring after the first date-of-service and having a negative result indicator of at least one of RAS, KRAS, or NRAS and to eliminate the first observation data record and the second observation data record as failing to satisfy the inter-date filter.

According to certain embodiments, the inter-date filter is configured to identify a first observation data record having a first date-of-service having a positive result indicator of at least one NRAS or KRAS and a second observation data record having a second date-of-service occurring after the first-date-of-service and having a negative result indicator of RAS and to eliminate the first observation data record and the second observation data record as failing to satisfy the inter-date filter. Moreover, the inter-date filter may be configured to identify a first observation data record having a first date-of-service and having a positive result indicator of at least one of RAS, KRAS, or NRAS and having a second observation data record having a second date-of-service occurring after the first date-of-service and having a negative result indicator of at least one of KRAS or NRAS and to eliminate the second observation data record as failing to satisfy the output filter. In certain embodiments, generating a model input data set is performed in accordance with a relevant data pre-processing methodology relating to colon cancer or rectal cancer, and wherein the method further comprises retrieving the relevant data pre-processing methodology from a plurality of data pre-processing methodologies based at least in part on the plurality of independently generated observation data records prior to generating the model input data set. In certain embodiments, the intra-date filter is configured to eliminate one or more observation data records failing to satisfy the intra-date filter relating to one of RAS, KRAS, or NRAS; and the inter-date filter is configured to eliminate one or more observation data records failing to satisfy the inter-date filter relating to one of RAS, KRAS, or NRAS. According to various embodiments, the computer-readable program code portions are further configured to apply a preliminary filter criteria before generating the model input data set, wherein the preliminary filter criteria comprise one or more of: a date-based filter criterion for selecting independently generated observation data records for further analysis as generated within a defined date range; a data source filter criterion for selecting independently generated observation data records for further analysis as generated by one or more defined data sources; or a data content filter criterion for selecting independently generated observation data records for further analysis as containing an identifier selected from a plurality of available identifiers eligible for further analysis. In certain embodiments, the machine-learning severity model is a linear regression model.

### BRIEF DESCRIPTION OF THE SEVERAL VIEWS OF THE DRAWINGS

Reference will now be made to the accompanying drawings, which are not necessarily drawn to scale, and wherein:

FIG. 1 is an exemplary overview of a system architecture that can be used to practice various embodiments;

FIG. 2 is an example schematic of a management computing entity in accordance with certain embodiments;

FIG. 3 is an example schematic of a user computing entity in accordance with certain embodiments;

FIG. 4 is a flowchart illustrating a general input process for intaking data in accordance with certain embodiments;

FIG. 5 is a flowchart illustrating an example pre-processing methodology for identifying relevant data records according to certain embodiments;

FIGS. 6A-6B show a flowchart illustrating an example process for identifying and rectifying intra-date conflicts between data records according to certain embodiments;

FIGS. 7A-7B show a flowchart illustrating a process for identifying and rectifying inter-date conflicts between data records according to certain embodiments; and

FIGS. 8A-8B show a flowchart illustrating a process for identifying and rectifying conflicts between data records according to certain embodiments.

### DETAILED DESCRIPTION

The present disclosure more fully describes various embodiments with reference to the accompanying drawings. It should be understood that some, but not all, embodiments are shown and described herein. Indeed, the embodiments may take many different forms, and accordingly this disclosure should not be construed as limited to the embodiments set forth herein. Rather, these embodiments are provided so that this disclosure will satisfy applicable legal requirements. Like numbers refer to like elements throughout.

### Overview

Clinical data can provide highly beneficial information about a patient's cancer which cannot be gleaned from claims data, such as data indicative of a cancer stage and biomarker status. However, observation data records (and/or other clinical data records) may be inconsistent, owing at least in part to the independent nature of each interaction between the patient and healthcare services (e.g., differing cancer stage values entered in the Electronic Health Record (EHR) by different providers or differing biomarker mutation results occurring on the same date or over time). These inconsistencies may impact the performance (e.g., accuracy and/or processing speed) of computer-based analytics, such as machine-learning based severity models that utilize observation data as a part of training data. The inconsistencies in observation data may impact the generation and/or training of machine-learning models, such that later implementation of the trained models is incapable of generating precise data outputs that can be relied upon for user decision making. As a result, there is a need for pre-processing of observation data to identify and/or reconcile any data conflicts which may exist for a compiled data set for a particular patient. Moreover, where these data sets are to be utilized as input for an analytical model, such as a machine-learning based severity model, data inconsistencies within a raw data set may cause the analytical model to output inaccurate analytical model results. Accordingly, various embodiments provide tailored pre-processing methodologies for intaking clinical data (e.g., data stored within a patient's EHR, data stored within one or more claims submitted to a healthcare payer, lab data indicative of lab test results, and/or the like) generated for each of a plurality of healthcare service interactions for the patient, and for generating a data set free of conflicting data that may be utilized in downstream analytics including, but not limited to, computer-based machine-learning modeling. This data may, in certain instances, provide an indication of expected cost related to treatment for the patient's colon or rectal cancer (e.g., an injectable chemotherapy) based on the detected presence or absence of RAS mutations within the patient's cancer.

### Technical Problem

A complete set of data generated for a patient's cancer diagnosis and treatment often includes one or more inconsistent data records, even when the cancer diagnosis has already been limited to a particular cancer type (e.g., colon cancer or rectal cancer), owing at least in part to the independent nature of each interaction between the patient and healthcare services (e.g., differing RAS mutation status results entered on the same date or over time). Regardless of the source and reason of the resulting inconsistent data records, these inconsistencies create difficulties for automated, computer-based models (e.g., machine-learning models) to generate accurate and relevant output. Particularly for machine learning models that utilize retrospective data sets as training data for the machine-learning models, these inconsistencies between data records may result in inaccurately trained machine learning models that do not optimally identify and model aspects of a cancer diagnosis, such as for identifying accurate cost estimates for treatment of a particular patient's cancer diagnosis.

### Technical Solution

To address the technical challenges presented by inconsistent data records existing within a data set related to a patient's cancer diagnosis and treatment, embodiments as discussed herein implement pre-processing methodologies for identifying and rectifying data inconsistencies within a data set for a particular patient, specifically RAS mutation status. In certain embodiments, a pre-processing system may be configured for executing one of a plurality of available pre-processing methodologies selected specifically to address the observation data records (and the included data elements) presented to the pre-processing system. The pre-processing methodologies discussed herein are specifically configured to identify and rectify inconsistencies in the identification of specific biomarker mutations within rectal cancer and colon cancer—those specific mutations encompassing RAS mutations and the more specific mutations thereof (KRAS and NRAS). These pre-processing methodologies are configured to identify and rectify inconsistencies within a data set encompassing a plurality of independently generated data records by excluding certain data records, generating additional data and/or metadata to be associated with particular data records as data tags, and/or the like. The pre-processing methodologies are executed by a management computing entity capable of providing the resulting pre-processed data as input directly to one or more downstream analytics, such as one or more downstream machine-learning based models, such as a severity model for generating severity data (e.g., estimated treatment costs) for a patient's cancer. Embodiments as discussed herein ensure that the pre-processing methodologies rectify identified data inconsistencies while minimizing data loss through filtering or other data exclusion by sequential application of data pre-processing methodologies and/or subprocesses that provide an increasingly clean data set as pre-processing subprocesses are applied.

### Computer Program Products, Methods, and Computing Devices

Embodiments of the present invention may be implemented in various ways, including as computer program products that comprise articles of manufacture. Such computer program products may include one or more software components including, for example, software objects, methods, data structures, and/or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present invention may also be implemented as methods, apparatus, systems, computing devices, computing entities, and/or the like. As such, embodiments of the present invention may take the form of a data structure, apparatus, system, computing device, computing entity, and/or the like executing instructions stored on a computer-readable storage medium to perform certain steps or operations. Thus, embodiments of the present invention may also take the form of an entirely hardware embodiment, an entirely computer program product embodiment, and/or an embodiment that comprises combination of computer program products and hardware performing certain steps or operations.

Embodiments of the present invention are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### Exemplary System Architecture

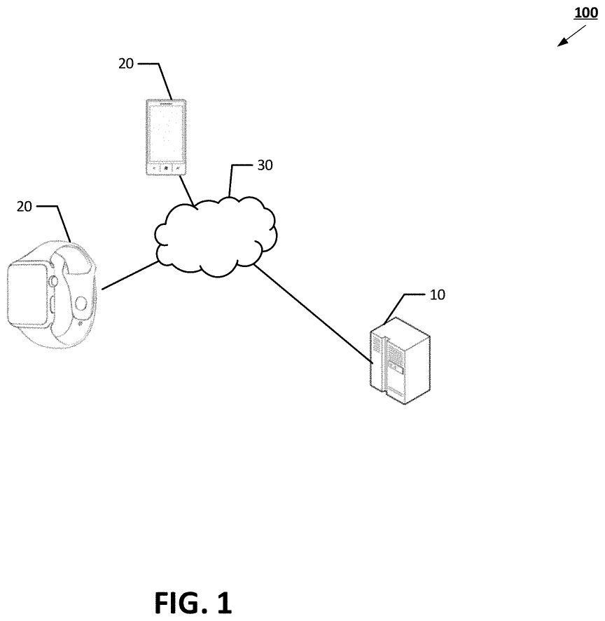

FIG. 1 provides an example system architecture 100 that can be used in conjunction with various embodiments of the present invention. As shown in FIG. 1, the system architecture 100 may comprise one or more management computing entities 10, one or more user computing entities 20, one or more networks 30, and/or the like. Each of the components of the system may be in electronic communication with, for example, one another over the same or different wireless or wired networks 30 including, for example, a wired or wireless Personal Area Network (PAN), Local Area Network (LAN), Metropolitan Area Network (MAN), Wide Area Network (WAN), and/or the like. Additionally, while FIG. 1 illustrates certain system devices as separate, standalone devices, the various embodiments are not limited to this particular architecture.

### Exemplary Management Computing Entity

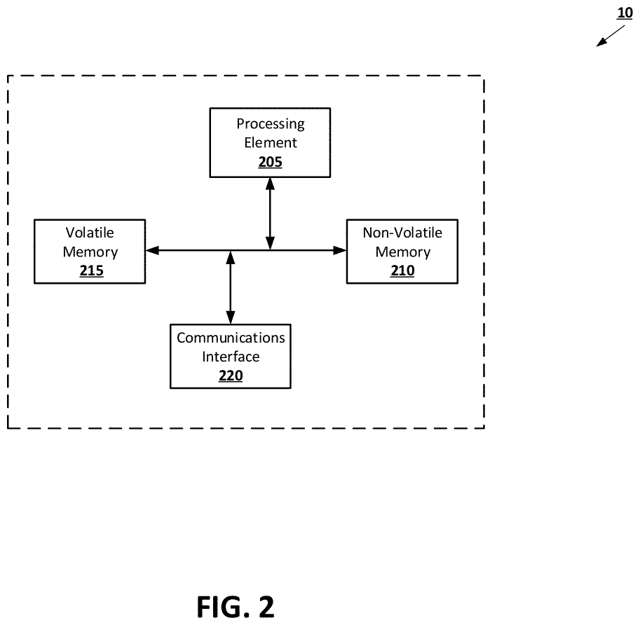

FIG. 2 provides a schematic of a management computing entity 10 according to one embodiment of the present invention. In general, the terms computing device, entity, device, system, and/or similar words used herein interchangeably may refer to, for example, one or more computers, computing devices, computing entities, desktop computers, mobile phones, tablets, phablets, notebooks, laptops, distributed systems, terminals, servers or server networks, blades, gateways, switches, processing devices, set-top boxes, relays, routers, network access points, base stations, the like, and/or any combination of devices adapted to perform the functions, operations, and/or processes described herein. Such functions, operations, and/or processes may include, for example, transmitting, receiving, operating on, processing, displaying, storing, determining, generating/creating, monitoring, evaluating, comparing, and/or similar terms used herein interchangeably. In one embodiment, these functions, operations, and/or processes can be performed on data, content, information, and/or similar terms used herein interchangeably.

As indicated, in one embodiment, the management computing entity 10 may also include one or more network and/or communications interfaces 220 for communicating with various computing entities, such as by communicating data, content, information, and/or similar terms used herein interchangeably that can be transmitted, received, operated on, processed, displayed, stored, and/or the like.

As shown in FIG. 2, in one embodiment, the management computing entity 10 may include or be in communication with one or more processing elements 205 (also referred to as processors, processing circuitry, and/or similar terms used herein interchangeably) that communicate with other elements within the management computing entity 10 via a bus, for example. As will be understood, the processing element 205 may be embodied in a number of different ways. For example, the processing element 205 may be embodied as one or more complex programmable logic devices (CPLDs), microprocessors, multi-core processors, coprocessing devices, application-specific instruction-set processors (ASIPs), and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like. As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present invention when configured accordingly.

In one embodiment, the management computing entity 10 may further include or be in communication with non-volatile media (also referred to as non-volatile storage, memory, memory storage, memory circuitry and/or similar terms used herein interchangeably). In one embodiment, the non-volatile storage or memory may include one or more non-volatile storage or memory media 210 as described above, such as hard disks, ROM, PROM, EPROM, EEPROM, flash memory, MMCs, SD memory cards, Memory Sticks, CBRAM, PRAM, FeRAM, RRAM, SONOS, racetrack memory, and/or the like. As will be recognized, the non-volatile storage or memory media may store databases, database instances, database management system entities, data, applications, programs, program modules, scripts, source code, object code, byte code, compiled code, interpreted code, machine code, executable instructions, and/or the like. The term database, database instance, database management system entity, and/or similar terms used herein interchangeably may refer to a structured collection of records or information/data that is stored in a computer-readable storage medium, such as via a relational database, hierarchical database, and/or network database.

In one embodiment, the management computing entity 10 may further include or be in communication with volatile media (also referred to as volatile storage, memory, memory storage, memory circuitry and/or similar terms used herein interchangeably). In one embodiment, the volatile storage or memory may also include one or more volatile storage or memory media 215 as described above, such as RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like. As will be recognized, the volatile storage or memory media may be used to store at least portions of the databases, database instances, database management system entities, data, applications, programs, program modules, scripts, source code, object code, byte code, compiled code, interpreted code, machine code, executable instructions, and/or the like being executed by, for example, the processing element 205. Thus, the databases, database instances, database management system entities, data, applications, programs, program modules, scripts, source code, object code, byte code, compiled code, interpreted code, machine code, executable instructions, and/or the like may be used to control certain aspects of the operation of the management computing entity 10 with the assistance of the processing element 205 and the operating system.

As indicated, in one embodiment, the management computing entity 10 may also include one or more network and/or communications interfaces 220 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, management computing entity 10 may be configured to communicate via wireless external communication networks using any of a variety of protocols, such as general packet radio service (GPRS), Universal Mobile Telecommunications System (UMTS), Code Division Multiple Access 200 (CDMA200), CDMA200 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), IR protocols, NFC protocols, RFID protocols, IR protocols, ZigBee protocols, Z-Wave protocols, 6LoWPAN protocols, Wibree, Bluetooth protocols, wireless universal serial bus (USB) protocols, and/or any other wireless protocol. The management computing entity 10 may use such protocols and standards to communicate using Border Gateway Protocol (BGP), Dynamic Host Configuration Protocol (DHCP), Domain Name System (DNS), File Transfer Protocol (FTP), Hypertext Transfer Protocol (HTTP), HTTP over TLS/SSL/Secure, Internet Message Access Protocol (IMAP), Network Time Protocol (NTP), Simple Mail Transfer Protocol (SMTP), Telnet, Transport Layer Security (TLS), Secure Sockets Layer (SSL), Internet Protocol (IP), Transmission Control Protocol (TCP), User Datagram Protocol (UDP), Datagram Congestion Control Protocol (DCCP), Stream Control Transmission Protocol (SCTP), HyperText Markup Language (HTML), and/or the like.

As will be appreciated, one or more of the management computing entity's components may be located remotely from other management computing entity 10 components, such as in a distributed system. Furthermore, one or more of the components may be aggregated and additional components performing functions described herein may be included in the management computing entity 10. Thus, the management computing entity 10 can be adapted to accommodate a variety of needs and circumstances, such as including various components described with regard to a mobile application executing on the user computing entity 20, including various input/output interfaces.

### Exemplary User Computing Entity

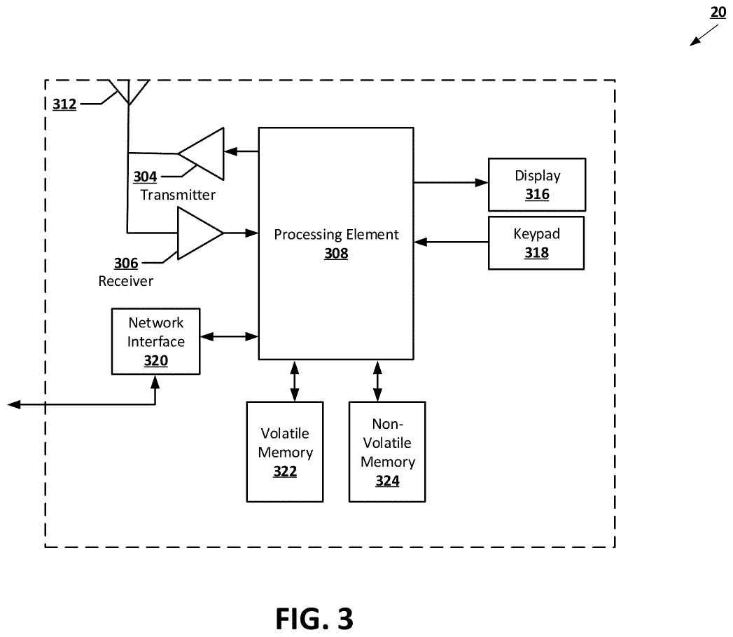

FIG. 3 provides an illustrative schematic representative of user computing entity 20 that can be used in conjunction with embodiments of the present invention. In various embodiments, the user computing entity 20 may be or comprise one or more mobile devices, wearable computing devices, and/or the like.

As shown in FIG. 3, a user computing entity 20 can include an antenna 312, a transmitter 304 (e.g., radio), a receiver 306 (e.g., radio), and a processing element 308 that provides signals to and receives signals from the transmitter 304 and receiver 306, respectively. The signals provided to and received from the transmitter 304 and the receiver 306, respectively, may include signaling information/data in accordance with an air interface standard of applicable wireless systems to communicate with various devices, such as a management computing entity 10, another user computing entity 20, and/or the like. In an example embodiment, the transmitter 304 and/or receiver 306 are configured to communicate via one or more SRC protocols. For example, the transmitter 304 and/or receiver 306 may be configured to transmit and/or receive information/data, transmissions, and/or the like of at least one of Bluetooth protocols, low energy Bluetooth protocols, NFC protocols, RFID protocols, IR protocols, Wi-Fi protocols, ZigBee protocols, Z-Wave protocols, 6LoWPAN protocols, and/or other short range communication protocol. In various embodiments, the antenna 312, transmitter 304, and receiver 306 may be configured to communicate via one or more long range protocols, such as GPRS, UMTS, CDMA200, 1×RTT, WCDMA, GSM, EDGE, TD-SCDMA, LTE, E-UTRAN, EVDO, HSPA, HSDPA, Wi-Fi, Wi-Fi Direct, WiMAX, and/or the like. The user computing entity 20 may also include one or more network and/or communications interfaces 320 for communicating with various computing entities, such as by communicating data, content, information, and/or similar terms used herein interchangeably that can be transmitted, received, operated on, processed, displayed, stored, and/or the like.

In this regard, the user computing entity 20 may be capable of operating with one or more air interface standards, communication protocols, modulation types, and access types. More particularly, the user computing entity 20 may operate in accordance with any of a number of wireless communication standards and protocols. In a particular embodiment, the user computing entity 20 may operate in accordance with multiple wireless communication standards and protocols, such as GPRS, UMTS, CDMA200, 1×RTT, WCDMA, TD-SCDMA, LTE, E-UTRAN, EVDO, HSPA, HSDPA, Wi-Fi, WiMAX, UWB, IR protocols, Bluetooth protocols, USB protocols, and/or any other wireless protocol.

Via these communication standards and protocols, the user computing entity 20 can communicate with various other devices using concepts such as Unstructured Supplementary Service information/data (USSD), Short Message Service (SMS), Multimedia Messaging Service (MMS), Dual-Tone Multi-Frequency Signaling (DTMF), and/or Subscriber Identity Module Dialer (SIM dialer). The user computing entity 20 can also download changes, add-ons, and updates, for instance, to its firmware, software (e.g., including executable instructions, applications, program modules), and operating system.

According to one embodiment, the user computing entity 20 may include location determining aspects, devices, modules, functionalities, and/or similar words used herein interchangeably to acquire location information/data regularly, continuously, or in response to certain triggers. For example, the user computing entity 20 may include outdoor positioning aspects, such as a location module adapted to acquire, for example, latitude, longitude, altitude, geocode, course, direction, heading, speed, UTC, date, and/or various other information/data. In one embodiment, the location module can acquire information/data, sometimes known as ephemeris information/data, by identifying the number of satellites in view and the relative positions of those satellites. The satellites may be a variety of different satellites, including LEO satellite systems, DOD satellite systems, the European Union Galileo positioning systems, the Chinese Compass navigation systems, Indian Regional Navigational satellite systems, and/or the like. Alternatively, the location information/data may be determined by triangulating the apparatus's 30 position in connection with a variety of other systems, including cellular towers, Wi-Fi access points, and/or the like. Similarly, the user computing entity 20 may include indoor positioning aspects, such as a location module adapted to acquire, for example, latitude, longitude, altitude, geocode, course, direction, heading, speed, time, date, and/or various other information/data. Some of the indoor aspects may use various position or location technologies including RFID tags, indoor beacons or transmitters, Wi-Fi access points, cellular towers, nearby computing entities (e.g., smartphones, laptops) and/or the like. For instance, such technologies may include iBeacons, Gimbal proximity beacons, BLE transmitters, NFC transmitters, and/or the like. These indoor positioning aspects can be used in a variety of settings to determine the location of someone or something to within inches or centimeters.

The user computing entity 20 may also comprise a user interface device comprising one or more user input/output interfaces (e.g., a display 316 and/or speaker/speaker driver coupled to a processing element 308 and a touch interface, keyboard, mouse, and/or microphone coupled to a processing element 308). For example, the user interface may be configured to provide a mobile application, browser, interactive user interface, dashboard, webpage, and/or similar words used herein interchangeably executing on and/or accessible via the user computing entity 20 to cause display or audible presentation of information/data and for user interaction therewith via one or more user input interfaces. Moreover, the user interface can comprise or be in communication with any of a number of devices allowing the user computing entity 20 to receive information/data, such as a keypad 318 (hard or soft), a touch display, voice/speech or motion interfaces, scanners, readers, or other input device. In embodiments including a keypad 318, the keypad 318 can include (or cause display of) the conventional numeric (0-9) and related keys (#, *), and other keys used for operating the user computing entity 20 and may include a full set of alphabetic keys or set of keys that may be activated to provide a full set of alphanumeric keys. In addition to providing input, the user input interface can be used, for example, to activate or deactivate certain functions, such as screen savers and/or sleep modes. Through such inputs the user computing entity 20 can capture, collect, store information/data, user interaction/input, and/or the like.

The user computing entity 20 can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, RRAM, SONOS, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like. The volatile and non-volatile storage or memory can store databases, database instances, database management system entities, information/data, applications, programs, program modules, scripts, source code, object code, byte code, compiled code, interpreted code, machine code, executable instructions, and/or the like to implement the functions of the user computing entity 20.

### Exemplary Networks

In one embodiment, any two or more of the illustrative components of the system architecture 100 of FIG. 1 may be configured to communicate with one another via one or more networks 30. The networks 30 may include, but are not limited to, any one or a combination of different types of suitable communications networks such as, for example, cable networks, public networks (e.g., the Internet), private networks (e.g., frame-relay networks), wireless networks, cellular networks, telephone networks (e.g., a public switched telephone network), or any other suitable private and/or public networks. Further, the networks 30 may have any suitable communication range associated therewith and may include, for example, global networks (e.g., the Internet), MANs, WANs, LANs, or PANs. In addition, the networks 30 may include any type of medium over which network traffic may be carried including, but not limited to, coaxial cable, twisted-pair wire, optical fiber, a hybrid fiber coaxial (HFC) medium, microwave terrestrial transceivers, radio frequency communication mediums, satellite communication mediums, or any combination thereof, as well as a variety of network devices and computing platforms provided by network providers or other entities.

### Example System Operation

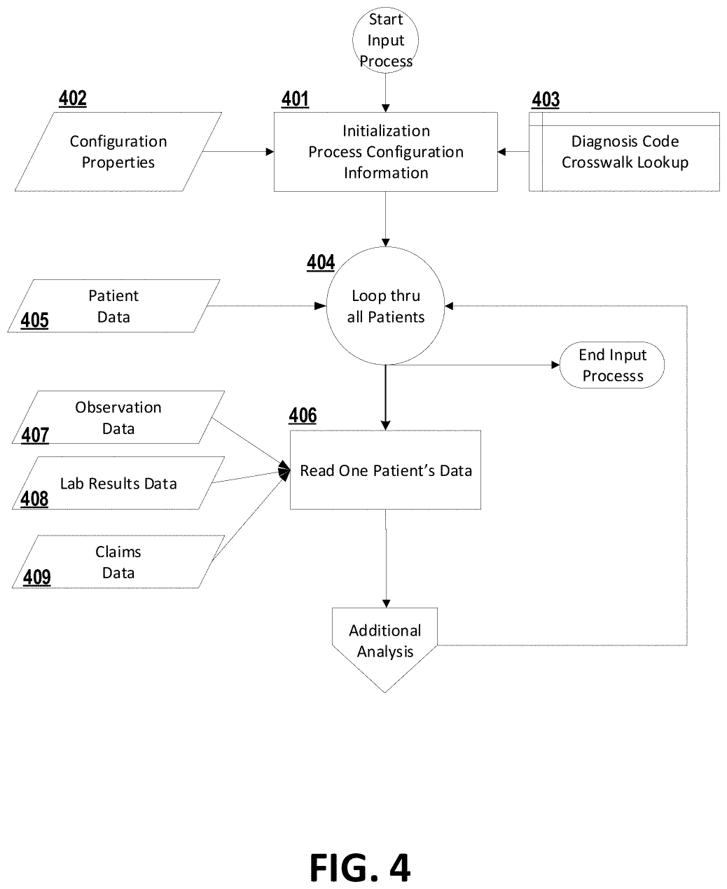

The pre-processing methodology of an example system operation is discussed below in reference to FIGS. 4-8B. The example operation of the overall system is discussed in terms of data inputs, data pre-processing, and output data to serve as input for further analytics, such as machine-learning based analytics. The configurations discussed herein are specifically provided for pre-processing colon cancer and rectal cancer data comprising data indicating the presence or absence of RAS, KRAS, and/or NRAS mutations within a patient's cancer cells, in order to generate consistent, clinically valid data which can subsequently be utilized in a plurality of analytical applications including, but not limited to computer-based modeling, such as machine-learning based severity models.

### Data Input

Certain embodiments encompass pre-processing configurations to provide pre-processing of data, such as claims data (e.g., data submitted to request reimbursement for medical services/products from a payer), and/or non-claims data, such as observation data (e.g., physician notes, prescription data, and/or the like) or other clinical data such as lab data, EHR data, data submitted during a prior authorization process, and/or the like to generate data sets capable of ingestion into a machine-learning based model for determining severity attributes of the model input data set, such as severity attributes of a patient's medical condition and/or medical treatment. In certain embodiments, the input data may be embodied as a plurality of data records, and each data record may comprise structured data embodied as a plurality of tagged data fields each having relevant data stored therein.

In certain embodiments, the input data corresponds with a particular patient (which may be identified by any of a variety of unique identifiers, such as a patient name, a patient unique user identifier, a unique identifier associated with the patient (e.g., a social security number), and/or the like. Moreover, the input data may be associated with one or more medical conditions, medical treatments, and/or the like, and such data may comprise a unique identifier indicative of the medical condition and/or medical treatment to which a particular data record (or other data grouping) applies. The unique identifier corresponding with the medical condition and/or medical treatment may be a universally recognized identifier taken from a universally known code-base, such as ICD codes (e.g., ICD-9 codes, ICD-10 codes, and/or the like). In certain embodiments, one or more unique identifiers associated with a medical condition may be embodied as a unique identifying code of a proprietary code set. It should be understood that the one or more lookup tables may comprise lookup tables provided as a part of an initial setup of the pre-processing system and/or one or more lookup tables provided by an end-user of the system (e.g., so as to enable the pre-processing system to operate properly with proprietary coding systems).

As indicated above, input data may be provided as one or more data records. In certain embodiments, each data record may correspond to a medical interaction with a patient. For example, such medical interactions may comprise an in-person visit between the patient and a medical professional, a virtual visit between the patient and a medical professional, a pharmaceutical prescription pick-up, a specific interaction with the patient during an in-patient stay at a medical facility, a medical device provided to the patient (e.g., as a prescription, as a part of an in-patient visit, and/or the like), a laboratory test performed on the patient, and/or the like.

In certain embodiments, the pre-processing system is configured to intake a plurality of data record types, such as claims data records, member data records, observation data records, other clinical data records (e.g., EHR data records, lab data records, and/or the like), and/or the like. The input data records may be provided with metadata defining one or more characteristics of the data record, thereby facilitating pre-processing thereof before providing the pre-processed data to one or more downstream analytics (such as a machine-learning severity model). Moreover, the pre-processed data may be stored and later merged with additional data to collectively define training data for one or more of the downstream analytics. For example, the pre-processed data may later be merged with externally-provided results data that provides an indication of one or more objective severity attributes (e.g., total incurred costs for treatment; itemized incurred costs for treatment; and/or the like) and the merged data may be utilized as training data for a machine-learning based severity model so as to enable generation of machine-learning severity models capable of generating accurate and precise outputs. The metadata may be provided together with the data file, or the metadata may be provided in a separate data file linked with (e.g., via matching reference identifiers) the underlying data record. The metadata may comprise data identifying, for example, a data record location (e.g., the storage location of the underlying data record), date formats within the data record, one or more dictionaries and/or reference tables for enabling automated processing of data elements within the data record.

The pre-processing systems are configured for filtering data records from a received collection of input data records such that removed data records are not provided to a downstream analytic model, such as an automated machine-learning based severity model, and accordingly the pre-processing system is configured to execute one or more data record validation processes, which are configured to ensure that the input data records are provided in a processable data format, comprise necessary data types, and/or the like. For example, the data record validation processes may be configured to ensure that each data record comprises one or more date fields (e.g., so as to ensure that only data records having corresponding dates falling within a defined date range are included; so as to enable chronological analysis of data records), and/or the like. In certain embodiments, the data record validation process is user configurable, and may comprise one or more configurable settings, such as defining pre-processing output storage locations, pre-processing log file storage locations, and/or the like.

The pre-processing data record validation processes are further configured to ensure that proper data and proper data records are provided as input before executing one or more pre-processing filtering processes. For example, the pre-processing data record validation processes of certain embodiments are configured to ensure that patient-identifying data records are provided along with one or more observation data records and/or claims data records. Accordingly, the pre-processing system is configured to intake patient-identifying data records along with one or more observation data records and/or claims data records. The pre-processing system performs such intake at a patient-level, such that observation data records and/or claims data records provided as input together with the patient identifying data record are associated with the patient identifying data record.

To intake the data, the pre-processing system reads in patient identifying data records to determine which observation data records are to be read for pre-processing. As mentioned, the observation data records contain a unique identifier corresponding to the patient, thereby enabling the patient data record to be matched with the observation data records.

Observation data records relating the patient data record are read from a clinical input file comprising a plurality of observation data records. As noted above, relevant observation data records are identified based at least in part on a patient identifier stored within the observation data records. Examples of observation data elements that may be contained within an observation data record encompass measurements of a patient's Body Mass Index (BMI), systolic and diastolic blood pressure (SBP and DBP), detections of RAS, KRAS, and/or NRAS mutations within a patient's colon cancer cells or rectal cancer cells (such data elements may encompass a plurality of data elements, such as identifying a biomarker mutation type and identifying whether the result indicator is positive or negative, which may be embodied as a positive result indicator or a negative result indicator, respectively), notes generated by a medical professional during an interaction with the patient, and/or the like. In accordance with certain embodiments, each observation data record contains only a single clinical measurement, and accordingly a single patient may have a plurality of observation data records generated within a single day. However, it should be understood that in certain embodiments, an observation data record may contain a plurality of measurements (e.g., a series of results, and/or the like) for a patient, and such observation data records may be subdivided as necessary to enable further pre-processing as discussed herein.

Lab data records relating to the patient data record are read from a lab results input file comprising a plurality of lab data records. As noted above, relevant lab data records are identified based at least in part on a patient identifier stored within the observation data records. Examples of lab data elements that may be contained within a lab data record encompass creatinine measurements, hemoglobin results, oncology biomarker mutation results (e.g., for detecting RAS/KRAS/NRAS mutations, including biomarker mutation indicators indicative of a mutation type and a separate positive or negative result indicator relating to the indicated mutation type), other oncology-related laboratory testing, and/or the like. In accordance with certain embodiments, each lab data record contains only a single lab measurement, and accordingly a single patient may have a plurality of lab data records generated within a single day. However, it should be understood that in certain embodiments, a lab data record may contain a plurality of lab measurements (e.g., results from a plurality of lab tests performed on a patient during a single day) for a patient, and such lab data records may be subdivided as necessary to enable further pre-processing as discussed herein.

Claims data records relating to the patient data record are read from one or more claims data sources. In certain embodiments, the claims data records may comprise claim data elements indicative of a medical procedure and/or a medical condition of the corresponding patient. Such data may be provided in the form of codes (e.g., proprietary codes, ICD-10 codes, and/or the like). In certain embodiments, the claims data records may be utilized to generate derived data elements for inclusion within an observation data record indicative of certain clinical observations and/or to resolve conflicts within and/or between observation data records for a particular patient. In certain embodiments, the derived data elements may each comprise a plurality of data elements, such as a derived observation identifier and a derived result indicator (positive or negative) for the derived observation identifier.

FIG. 4 is a flowchart illustrating an example process for intaking and processing data. As shown therein, the pre-processing system initializes the data input process as shown at Block 401, by ingesting configuration properties (shown at Block 402) for execution of the data input process. Moreover, as needed, one or more lookup tables may be referenced during initialization (e.g., if diagnosis codes are to be translated between coding structures, such as between a proprietary code set and ICD-10 codes) as indicated at Block 403.

The process is performed one patient at a time, for all patients for which patient data is provided (as indicated at Blocks 404-405 and the looping structure of the flowchart). For each patient, observation data records, lab data records, other clinical data records, claims data records, and/or any other data records relevant to a member are read, as indicated at Blocks 406-409.

The data intake process may proceed in accordance with one or more configuration properties defined for a pre-processing system that executes the various pre-processing rules (e.g., including data content filter criteria reviewing the content of structured observation data records, data source filter criteria reviewing the source of a structured observation data record, a date-based filter criteria, and/or the like) prior to passing pre-processed data to an analytical model, such as a machine-learning based severity model. Moreover, the pre-processing system may reference one or more lookup tables stored in a memory storage area accessible to the pre-processing system.

The process is performed for all patients for which patient data is provided. For each patient, observation data records, patient records, claims data records, and/or any other data records relevant to a particular patient are read to enable pre-processing thereof.

In certain embodiments, each of the patient data records and/or the observation data records may be stored within a relational database, with each data record stored separately, and having one or more data elements stored therein that may be utilized to identify relationships between data records (e.g., via a unique patient identifier). However, it should be understood that other database structures may be utilized in certain embodiments.

In example embodiments, the patient data files comprise a unique patient identifier and a date of birth for the patient. The patient data files may comprise additional data relevant to a patient, such as contact information (e.g., a phone number, an email address, a home address, a mailing address, and/or the like), insurance information (e.g., patient eligibility data, such as identifying a health insurance provider, health insurance membership plan information, and/or the like), and/or the like.

Observation data records comprise, for example, a unique patient identifier to enable association with a patient data record, a unique record identifier that may be utilized to quickly identify a particular observation record, concept type data (indicating the type of data stored within the observation record, such as a particular biomarker mutation, cancer stage, and/or the like, which may be indicated via proprietary codes from a proprietary code set established as a taxonomy of various concepts), a start date for the observation, a biomarker mutation identifier (e.g., indicative of the specific biomarker mutation relevant to the observation data record, such as RAS, KRAS, or NRAS), a result indicator indicating whether the observation data record reflects a “positive” or “negative” result for the particular biomarker mutation, such as RAS, KRAS, or NRAS, and/or the like), a concept condition identifier (a condition for which the observation data record is provided; in oncology-related measurements the concept condition identifier indicates a cancer type), a data source identifier (indicative of the source from which the observation data record is retrieved, such as a patient's electronic medical record, a pre-authorization request data record submitted to an insurance payer, and/or the like). As discussed herein, pre-processing filter rules may be data content based, such as for filtering observation data records based at least in part on data content stored within one or more data fields, such as within the biomarker mutation data. Pre-processing filter rules may additionally or alternatively encompass data source-based filtering rules for filtering data based on a generating data source (as discussed herein). Other pre-processing filter rules may encompass date-based filter rules for filtering data records based at least in part on date data stored therein.

Lab data records of certain embodiments comprise a unique patient identifier to enable association with a patient data record, a unique record identifier that may be utilized to quickly identify a particular lab data record, a code type (that may be utilized to indicate the taxonomy of the additional data elements provided in the lab data record, such as indicating whether the lab results data record utilizes a coding structure such as a Logical Observation Identifiers Names and Codes (LOINC) coding structure, a proprietary coding structure of a particular lab, and/or the like), a code (e.g., a LOINC code indicative of a lab test performed), a start date for the lab result, a data element indicative of the lab result (e.g., a numeric data element, a non-numeric data element, a binary data element, and/or the like), a results unit identifier (indicative of the units relevant to the lab result itself), a data source identifier indicative of the source from which the observation data record is retrieved (e.g., a patient's electronic medical record, a pre-authorization request data record submitted to an insurance payer, and/or the like), and/or the like. It should be understood that in certain embodiments, a lab data record may encompass observation data encompassing a biomarker mutation identifier and a result indicator analogous to the observation data records discussed above.

Claims data records of certain embodiments comprise a unique patient identifier enabling association with a patient data record, a code type (that may be utilized to indicate the taxonomy of the additional data elements provided in the claims data record, such as indicating whether the claims data record utilizes ICD-10 codes, ICD-9 codes, or another diagnosis code taxonomy), one or more diagnosis codes, a first date of service associated with the claim, a last date of service associated with the claim, and/or a unique record identifier that may be utilized to quickly identify a particular claim data record.

### Data Pre-Processing

As discussed herein, the data pre-processing methodologies may be configured for pre-processing observation data records, lab data records, and/or the like, having concept type data encompassing a biomarker mutation indicator and a result indicator indicative of the presence or absence of specific biomarker mutations detected within a patient's colon cancer cells or rectal cancer cells. The concept type data (inclusive of the biomarker mutation indicator) sourced from clinical observations provides information regarding certain characteristics of a patient's colon cancer or rectal cancer that may indicate whether particular treatment types would be effective against the patient's cancer and/or may indicate an estimated treatment cost for the patient's cancer. Such observation data cannot be replicated from claims data alone. Moreover, this information can be used to better understand the associated estimated treatment costs. In certain embodiments, observation data records comprising biomarker mutation data may be collected during a prior authorization process for claims submissions and/or by collecting data from a patient's EHR.

The pre-processing methodology discussed herein is configured to output clinically valid data records having consistent biomarker mutation results for a single patient over a defined time frame. The pre-processing system thus identifies clinically invalid and/or conflicting biomarker mutation results within observation data records, such that only consistent data records constituting valid and non-conflicting findings are provided to downstream analytics (e.g., machine-learning based severity models). Consistent observation data records as discussed herein are defined as a plurality of observation data records that either remain constant over time or demonstrate a natural progression of the development of RAS mutations within colon cancer cells or rectal cancer cells, as determined to be clinically valid. The natural progression of the development of RAS mutations indicates that RAS mutations may develop over time, but the RAS mutations cannot naturally disappear from a patient's cancer cells. In other words, a plurality of observation data records indicating that a chronologically first observation data record that does not demonstrate the presence of RAS mutations, followed by a chronologically second observation data record that demonstrates the presence of RAS mutations is considered valid. By contrast, a plurality of observation data records indicating that a chronologically first data record that demonstrates the presence of RAS mutations, followed by a chronologically second observation data record that does not demonstrate the presence of RAS mutations is considered invalid. By extension, multiple observation data records generated for a single day that includes inconsistent biomarker mutation results (e.g., one observation data record indicating the presence of RAS mutations and a second observation data record indicating the absence of RAS mutations) are considered invalid.

As discussed herein, biomarker mutation indicators may provide indications of more specific RAS mutations, such as NRAS and/or KRAS mutations, and therefore the pre-processing methodology additionally ensures that observation data records satisfy applicable consistency rules and filters across biomarker mutation indicators. Such rules and filters ensure that that biomarker mutation results reflective of the presence of KRAS or NRAS mutations are not contradicted by a negative RAS mutation (as noted above, KRAS and NRAS are more specific forms of RAS mutations), for example.

Rules and filters for identifying observation data records containing consistent biomarker mutation indicators may be included within a single model or multiple consecutively executed models for identifying and/or rectifying various inconsistencies within a data set. The rules and filters may encompass intra-date rules and filters for identifying and rectifying inconsistent observation data records occurring within a single day and/or inter-day rules and filters for identifying and rectifying inconsistent observation data records occurring across multiple days (e.g., within a defined time period).

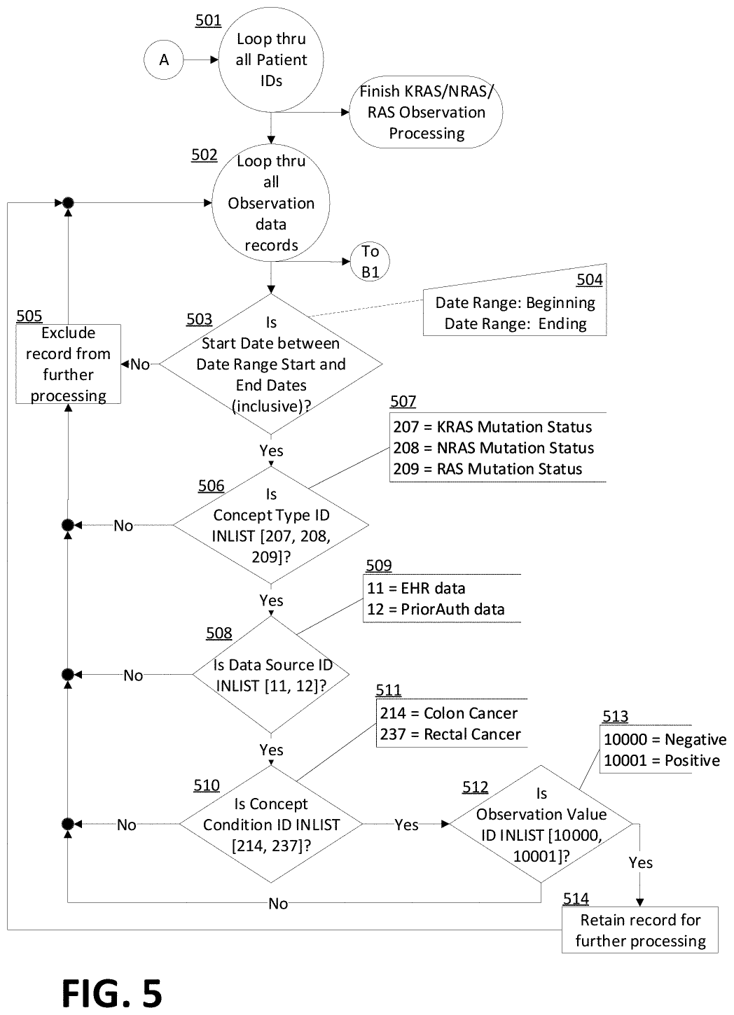

With reference to FIGS. 5-8B, which illustrates various flowcharts indicative of processes for applying rules and filters to observation data records, certain embodiments are configured to begin by applying rules and filters for selecting relevant observation data records for further analysis, as indicated in FIG. 5. As indicated at Block 501, the processes for executing rules and filters is repeated for all patients (based on unique patient identifying data, as discussed above), and as indicated at Block 502, all observation data records relevant to a particular patient are considered such that rules and filters are appropriately applied.

As shown at Block 503, the pre-processing methodology identifies observation data records having start dates identified as defining a relevant time frame for analysis. The date range may be defined within the configuration data for the pre-processing system in general and may be defined based at least in part via a beginning date and an ending date for the date range (as shown in Block 504). Those records having dates that do not fall within the relevant date range are excluded from further analysis, as indicated at Block 505.

The pre-processing methodology continues by identifying data records having concept type data indicating the observation data record relates to RAS/KRAS/NRAS biomarker mutation indicators, as indicated at Block 506 (with reference to a lookup table identifying concept type indicators for each of a RAS mutation status, a KRAS mutation status, and a NRAS mutation status, as indicated at Block 507). Those records that do not relate to these biomarker mutation indicators are excluded from further analysis, as indicated at Block 505. As noted above, such observation data records comprise concept type data encompassing a biomarker mutation indicator and a result indicator, respectively reflecting a biomarker mutation to which the observation data record relates.

The pre-processing methodology continues by identifying data records indicating the data record source is one of one or more permitted data sources, as indicated at Block 508. For example, the pre-processing methodology may be configured to maintain observation data records obtained from a patient's EHR and/or from prior authorization request data (as indicated at Block 509). In certain embodiments, the pre-processing methodology is configured to accept data records from a single permitted data source. Those observation data records indicated as received from an unpermitted data source are excluded from further analysis, as indicated at Block 505.

The pre-processing methodology continues by identifying data records indicating the data record relates to colon cancer or rectal cancer, as indicated at Block 510 (with reference to a lookup table identifying unique concept condition indicators for each of colon cancer and rectal cancer, as reflected at Block 511). Observation data records that do not relate to colon cancer or rectal cancer are excluded from further analysis, as indicated at Block 505.

To ensure that only observation data records having relevant information types relating to biomarker mutation indicators are present, the pre-processing methodology next determines whether data elements provided with respect to the biomarker mutation indicator encompass a result indicator comprising an accepted data content (e.g., a “positive” indication of an associated biomarker mutation or a “negative” indication of an associated biomarker mutation), such that appropriate analysis may be performed in accordance with expected data types. Such an analysis is reflected at Block 512 (with reference to a lookup table indicated at Block 513 providing a list of allowable data contents). Observation data records that contain other data within the result indicator field of the biomarker mutation indicator are excluded from further analysis, as indicated at Block 505. However, those records that are retained following the analysis provided in accordance with Block 512 are retained for additional pre-processing, as reflected at Block 514.

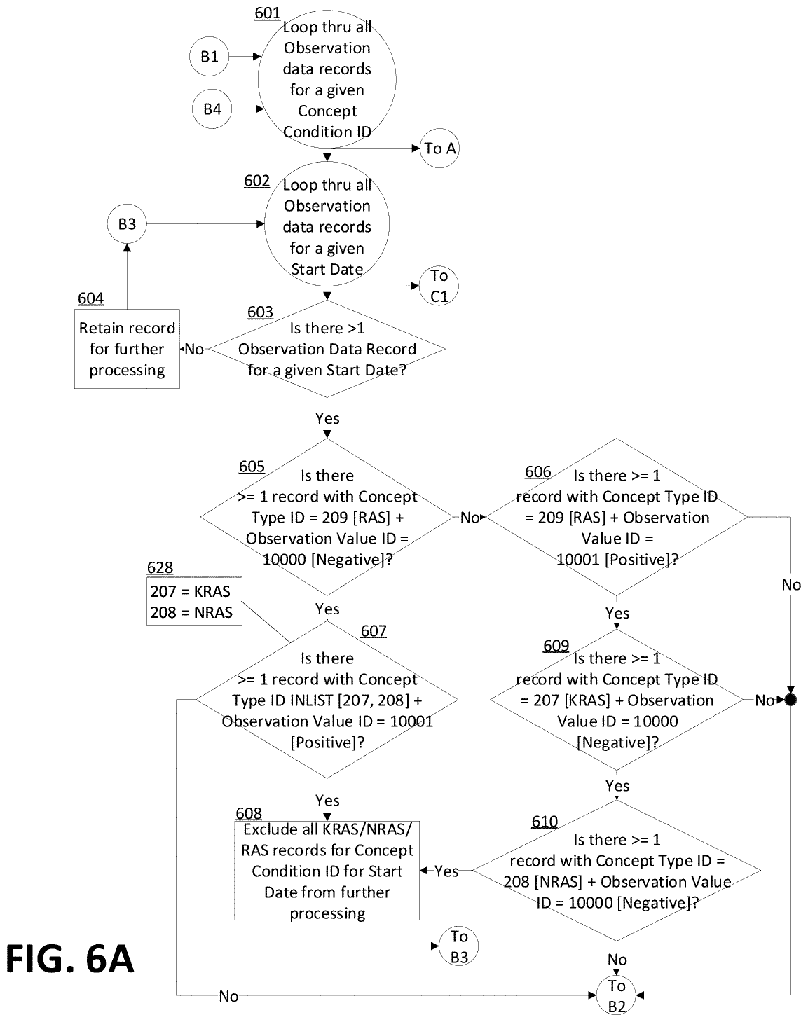

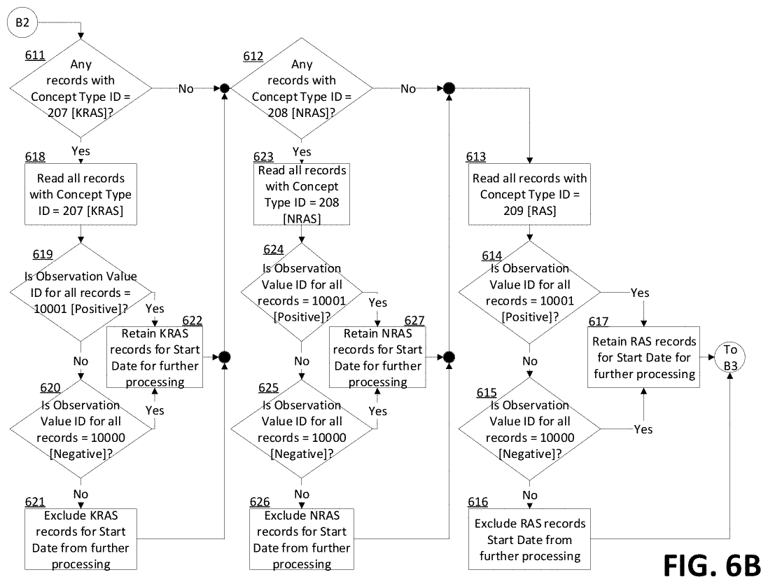

The pre-processing methodology continues by configuring the pre-processing system to apply various filtering analyses for identifying any identified conflicts between data records. The pre-processing system, executing the pre-processing methodology, first identifies intra-date conflicts in accordance with the example sub-process reflected in FIGS. 6A-6B. As indicated at Block 601, the process for identifying intra-date conflicts for a particular condition (e.g., a particular cancer type) is applied for all observation data records that were retained after the initial pre-processing sub-process discussed in reference to FIG. 5 (as reflected in Block 601). Moreover, intra-date conflicts are identified and rectified for each date within a time frame of interest for a patient, and therefore the described process is performed separately for each date, as reflected at Block 602. If the pre-processing system executing the pre-processing methodology identifies only one observation data record for a particular date (as shown at Block 603), the observation data record is retained for further analysis, as indicated at Block 604. However, if more than one observation data record is identified for a particular date, those identified observation data records are further analyzed to determine whether they should remain in the data set. As shown at Blocks 605-606, the pre-processing methodology causes the pre-processing system to determine whether any observation data records exist having RAS biomarker mutation indicators; the step of Block 605 determines whether there are any RAS negative observation data records, and the step of Block 606, which is considered if there are no RAS negative observation data records, determines whether there are any RAS positive observation data records. If there are neither RAS negative nor RAS positive observation data records, the process proceeds as discussed in reference to FIG. 6B.

However, if at Block 605, the pre-processing methodology causes the pre-processing system to determine that at least one observation data record exists for a particular date having a biomarker mutation indicator reflecting a RAS negative result indicator, the pre-processing methodology next causes the pre-processing system to determine whether any other observation data records encompass biomarker mutation indicator data records that conflict with the identified RAS negative result indicator detected for the particular date. As reflected at Block 607, the pre-processing methodology is configured to cause the pre-processing system to determine whether any KRAS positive or NRAS positive observation data records exist for the same date (with reference to a lookup table reflecting unique indicators for KRAS and NRAS, as reflected at Block 628). If there are no KRAS positive or NRAS positive records present for the particular date, the analysis proceeds as reflected in FIG. 6B. However, if at least one KRAS positive or NRAS positive record is identified for the particular date, all of the observation data records for the particular date and concept condition are excluded from further analysis, as indicated at Block 608.

If, at Block 606, the pre-processing system executing the pre-processing methodology determines that at least one RAS positive record is present for a particular date, the pre-processing system next determines whether any other biomarker mutation records conflict with the identified RAS positive result indicator for the particular date. As reflected at Block 609, the pre-processing system determines whether any KRAS negative result indicator-containing records exist for the particular date. If no KRAS negative result indicator-containing records exist for the particular date, the process proceeds in accordance with FIG. 6B. However, if a KRAS negative result indicator-containing record is identified, the pre-processing system next determines whether any NRAS negative result indicator-containing records exist for the particular date. In short, the pre-processing methodology ensures that there is not a RAS positive result indicator and both a KRAS negative result indicator and an NRAS negative result indicator for the particular date through the processes of Blocks 606, 609, 610. If, according to the pre-processing methodology, the pre-processing system determines that a particular date has a RAS positive result indicator and both NRAS negative result indicator and KRAS negative result indicators, all observation data records for that particular date and concept condition are excluded from further analysis, as reflected at Block 608. However, if the particular date does not include all of (a) RAS positive result indicator, (b) KRAS negative result indicator, and (c) NRAS negative result indicator, the process proceeds in accordance with FIG. 6B. The sub-processes of FIG. 6A ensure that no conflicts exist between different biomarker mutation indicator types (e.g., between RAS biomarker mutation indicators and KRAS biomarker mutation indicators or NRAS biomarker mutation indicators). The pre-processing system then determines whether any conflicts exist between observation data records of the same biomarker mutation indicator type (e.g., between observation data records having KRAS biomarker mutation indicators; between observation data records having NRAS biomarker mutation indicators; and/or between observation data records having RAS biomarker mutation indicators).

The data records that are retained for analysis under the additional intra-day conflict analysis of FIG. 6B are analyzed to determine what types of biomarker mutation indicators in the observation data records are present for the particular date, and whether those observation data records satisfy applicable conflict rules and filters. Initially, the observation data records for a particular date are analyzed to determine whether any KRAS biomarker mutation indictor observation data records are present, as indicated at Block 611, whether any NRAS biomarker mutation indicator observation data records are present, as indicated at Block 612, and whether any RAS biomarker mutation indicator observation data records are present, as indicated at Block 613. As referenced in Block 613, for dates in which RAS biomarker mutation indicator observation data records are present, the pre-processing methodology ensures that the RAS biomarker mutation indicator observation data records are all positive or all negative (as reflected at Blocks 614-615). If the RAS observation data records for that particular date are not all RAS positive or all RAS negative, all RAS data records are excluded for that date and concept condition, as reflected at Block 616. However, if the RAS observation data records are all RAS positive or all RAS negative for that particular date, all RAS observation data records for that date are retained for further analysis, as indicated at Block 617.

With reference again to Block 611, if at least one KRAS biomarker mutation indicator observation data record is identified, the process proceeds to Block 618, which indicates that all KRAS observation data records are read, and at Blocks 619-620, the pre-processing methodology ensures that all of the KRAS biomarker mutation indicator observation data records are all positive or all negative. If the KRAS records for that particular date are not all KRAS positive or all KRAS negative, all KRAS data records are excluded for that date and concept condition, as reflected at Block 621. However, if the KRAS records for that particular date are all KRAS positive or all KRAS negative, all KRAS records for that date are retained for further analysis, as indicated at Block 622. The process then continues to a similar analysis for NRAS data records, beginning with Block 612 as noted above. However, it should be noted that the analysis of RAS, KRAS, and NRAS data records may be reversed in accordance with certain embodiments.

If, according to Block 612, at least one NRAS observation data record is identified for a particular date, the process proceeds to Block 623, which indicates that all NRAS observation data records are read, and at Blocks 624-625, the pre-processing methodology ensures that the NRAS records for that particular date are all positive or all negative. If the NRAS observation data records for that particular date are not all NRAS positive or all NRAS negative, all NRAS data records are excluded for that particular date and concept condition, as reflected at Block 626. However, if the NRAS observation data records are all NRAS positive or all NRAS negative for that particular date, all NRAS observation data records for that date are retained for further analysis, as indicated at Block 627.

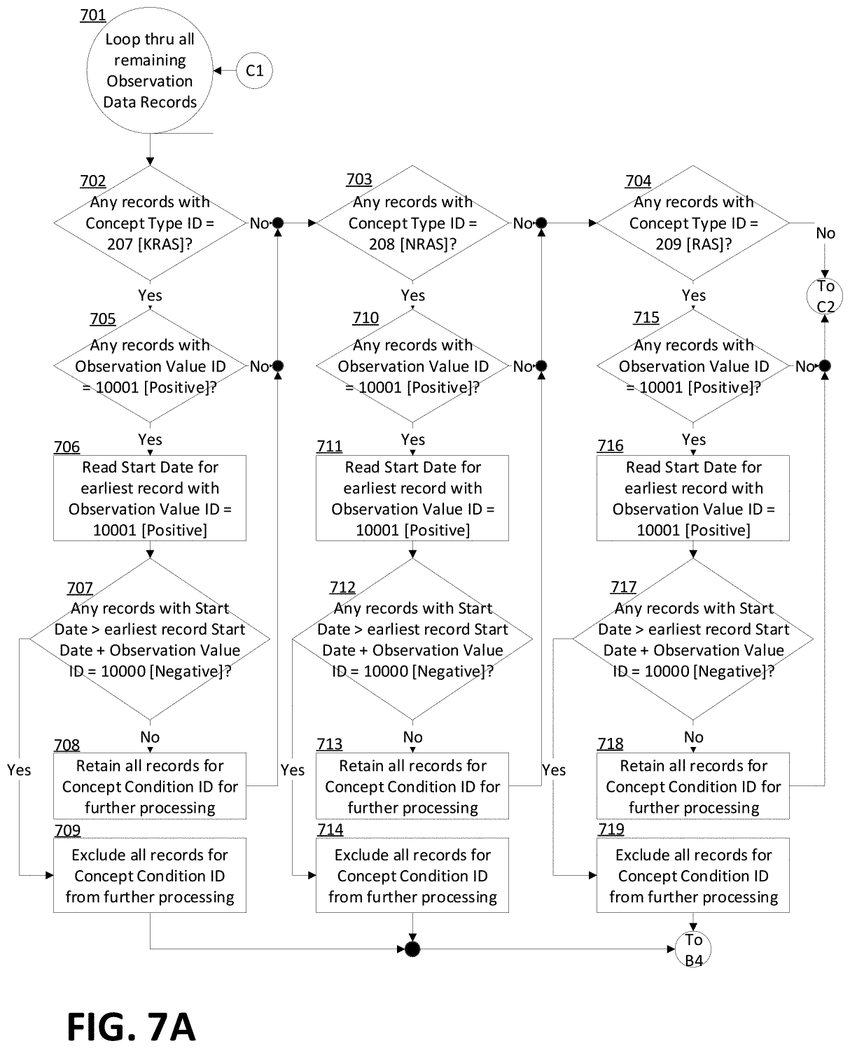

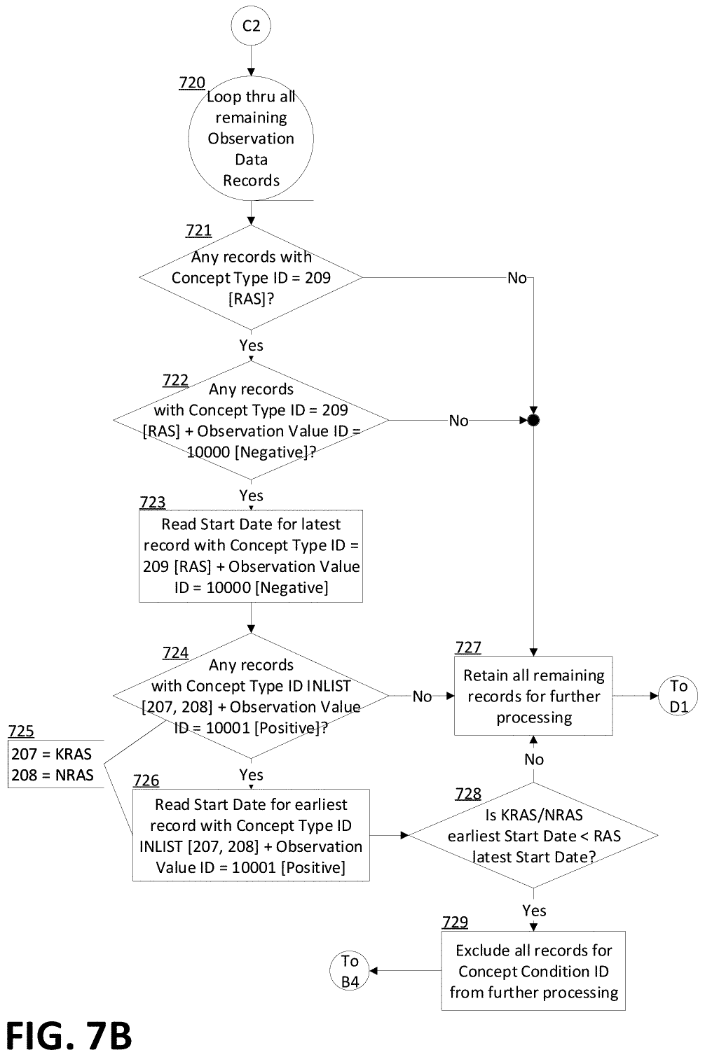

Those records that are retained after identifying and rectifying intra-date conflicts in accordance with FIGS. 6A-6B are further analyzed to identify and rectify inter-date conflicts in accordance with FIGS. 7A-7B (as indicated at Block 701). The processes reflected in each of FIGS. 7A-7B are executed with respect to all data records remaining within the data set. The analysis for inter-date conflicts may proceed based on biomarker mutation indicator data records identified for a particular patient and date range. Accordingly, through Blocks 702-704, the pre-processing system executing the pre-processing methodology consecutively determines whether KRAS biomarker mutation indicator records are present (as indicated at Block 702), then determines whether NRAS biomarker mutation indicator data records are present (as indicated at Block 703), and then determines whether RAS biomarker mutation indicator data records are present (as indicated at Block 704). For each biomarker mutation indicator data record identified, the pre-processing system completes a series of additional sub-processes before moving to the next identified biomarker mutation indicator data record. It should be understood that while Blocks 702-704 indicate one example order of identifying and analyzing biomarker mutation indicator data records (with KRAS biomarker mutation indicator records identified and analyzed first, followed by NRAS biomarker mutation indicator records, and lastly RAS biomarker mutation indicator records), other orders of analysis are possible in certain embodiments (e.g., beginning with NRAS biomarker mutation indicator records, followed by KRAS biomarker mutation indicator records, and followed by RAS biomarker mutation indicator records).

As indicated at Block 702, upon identifying KRAS biomarker mutation indicator data records for a particular time period, the pre-processing system determines whether any of the identified KRAS biomarker mutation indicator data records have a KRAS positive result indicator, as indicated at Block 705. If no KRAS positive result indicators are present, the pre-processing methodology proceeds to cause the pre-processing system to conduct a similar analysis of NRAS biomarker mutation indicators (beginning with Block 703). However, if at least one KRAS positive result indicator is present, the pre-processing system determines the start date for the earliest KRAS positive result indicator (as indicated at Block 706), and then determines whether there are any observation data records having a KRAS negative result indicator with a start date after the start date of the earliest KRAS positive result indicator, as indicated at Block 707. In other words, the pre-processing methodology determines whether the data set for the patient indicates a shift from a KRAS positive result indicator to a KRAS negative result indicator. Such a shift from KRAS positive to KRAS negative is deemed clinically invalid, and therefore all observation data records for the concept condition (including all KRAS, all NRAS, and all RAS observation data records) are excluded from further analysis, as indicated at Block 709, and analysis for that patient ends (in accordance with FIG. 7A). However, if there are no KRAS negative result indicators occurring after the start date of the earliest KRAS positive result indicator, all KRAS records are retained for further analysis, as indicated at Block 708, and the process proceeds to conduct an analogous analysis for NRAS biomarker mutation indicator observation data records.

With reference again to Block 703, upon identifying at least one NRAS biomarker mutation indicator observation data record for a particular time period, the pre-processing methodology determines whether any of the identified NRAS biomarker mutation indicator data records have an NRAS positive result indicator, as indicated at Block 710. If no NRAS positive result indicators are present, the pre-processing methodology proceeds to conduct a similar analysis of RAS biomarker mutation indicators (beginning at Block 704). However, if at least one NRAS positive result indicator is present, the pre-processing methodology causes the pre-processing system to determine the start date for the earliest NRAS positive result indicator (as indicated at Block 711) and determines whether there are any observation data records having an NRAS negative result indicator with a start date after the start date of the earliest NRAS positive result indicator, as indicated at Block 712. In other words, the pre-processing system determines whether the data set for the patient indicates a shift from an NRAS positive result indicator to an NRAS negative result indicator. Such a shift from NRAS positive to NRAS negative is deemed clinically invalid, and therefore all observation data records for the concept condition (including all KRAS, all NRAS, and all RAS observation data records) are excluded from further analysis, as indicated at Block 714, and analysis ends for that patient (in accordance with FIG. 7A). However, if there are no NRAS negative result indicators occurring after the start date of the earliest NRAS positive result indicator, all NRAS records are retained for further analysis, as indicated at Block 713, and the process proceeds to conduct an analogous analysis for RAS biomarker mutation indicator observation data records.

With reference again to Block 704, upon identifying at least one RAS biomarker mutation indicator observation data record for a particular time period, the pre-processing system executing the pre-processing methodology determines whether any of the identified RAS biomarker mutation indicator data records have a RAS positive result indicator, as indicated at Block 715. If no RAS positive result indicators are present, the pre-processing methodology continues in accordance with the further analysis reflected in FIG. 7B, discussed below. However, if at least one RAS positive result indicator is present, the pre-processing methodology causes the pre-processing system to determine the start date of the earliest RAS positive result indicator (as indicated at Block 716) and then determines whether there are any observation data records having a RAS negative result indicator with a start date after the start date of the earliest RAS positive result indicator, as indicated at Block 717. In other words, the pre-processing methodology causes the pre-processing system to determine whether the data set for the patient indicates a shift from a RAS positive result indicator to a RAS negative result indicator. Such a shift from RAS positive to RAS negative is deemed clinically invalid, and therefore all observation data records for the concept condition (including all KRAS, all NRAS, and all RAS observation data records) are excluded from further analysis, as indicated at Block 719, and analysis ends for that patient (in accordance with FIG. 7A). However, if there are no RAS negative result indicators occurring after the start date of the earliest RAS positive result indicator, all RAS records are retained for further analysis, as indicated at Block 718, and the process proceeds to conduct further analysis in accordance with FIG. 7B.

The sub-process illustrated in FIG. 7B ensures that inter-date conflicts between biomarker mutation indicator types (e.g., between a KRAS biomarker mutation indicator or NRAS biomarker mutation indicator of a first observation data record and a RAS biomarker mutation indicator of a second observation data record) are not present within the data set remaining after sub-processes as discussed in FIGS. 5-7A are executed with respect to the data set. Accordingly, as indicated at Block 720, the process illustrated in FIG. 7B is executed with respect to all data records remaining within the data set.

The analysis according to FIG. 7B begins by reviewing the remaining observation data records to determine whether any records have a RAS biomarker mutation indicator (as shown at Block 721). If none of the observation data records have a RAS biomarker mutation indicator, all observation data records are retained for further analysis, as indicated at Block 727.

However, if at least one RAS biomarker mutation indicator observation data record is identified, the pre-processing methodology continues by causing the pre-processing system to determine whether any of the identified data records containing RAS biomarker mutation indicators have a RAS negative result, as reflected at Block 722. If the pre-processing methodology determines that the RAS biomarker mutation indicators are all RAS positive, all the observation data records are retained for further processing, in accordance with Block 727.

However, if at least one RAS negative result indicator is identified, the latest RAS negative observation data record is identified and the pre-processing methodology retrieves the start date for this latest RAS negative observation data record as shown at Block 723. The pre-processing methodology next causes the pre-processing system to determine whether any NRAS positive or KRAS positive observation data records exist within the remaining data records (Block 724) and have a start date occurring before the start date of the latest RAS negative observation data record (as determined at Block 723). In other words, specifically at Block 724 (with reference to a lookup table as indicated at Block 725), the pre-processing system determines whether any KRAS positive or NRAS positive result indicators have a start date prior to the latest RAS negative result indicator within the remaining data set (Block 726 and 728). The transition from any positive mutation (e.g., KRAS positive or NRAS positive) to a RAS negative mutation over time is deemed clinically invalid, and thus data sets reflecting such a transition are deemed unreliable and all KRAS, NRAS, and RAS results for the concept condition for that patient are excluded from further analysis (Block 729). If the pre-processing methodology does not identify any KRAS positive or NRAS positive data records having a start date prior to the latest RAS negative data record, all observation data records are retained for further processing, as indicated at Block 727.

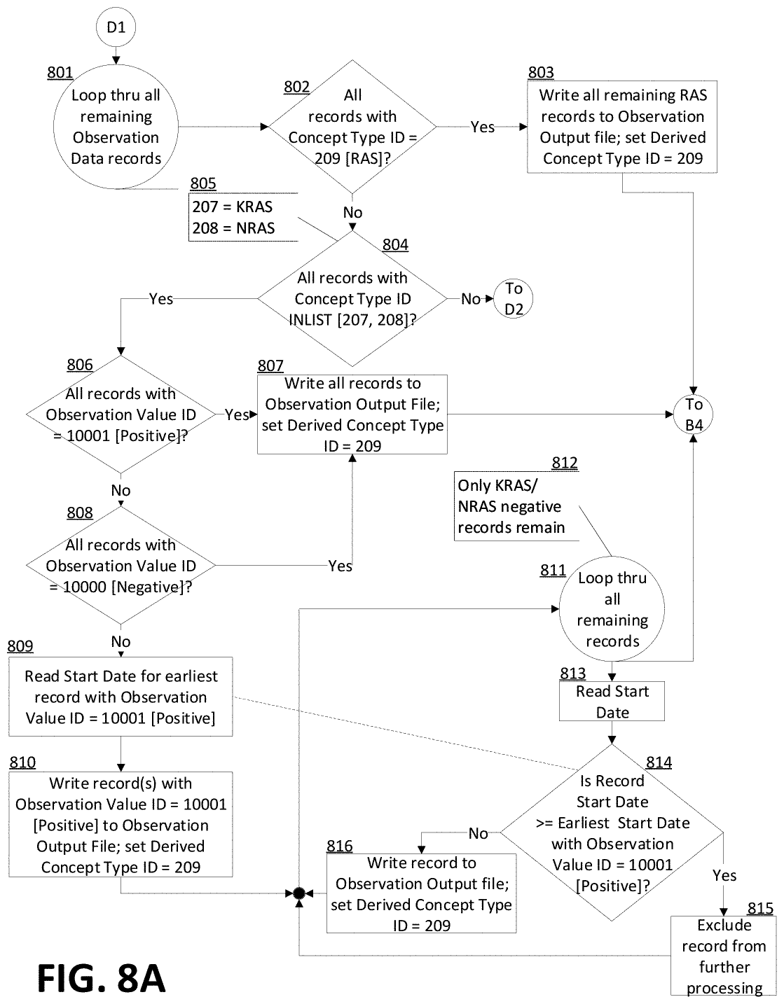

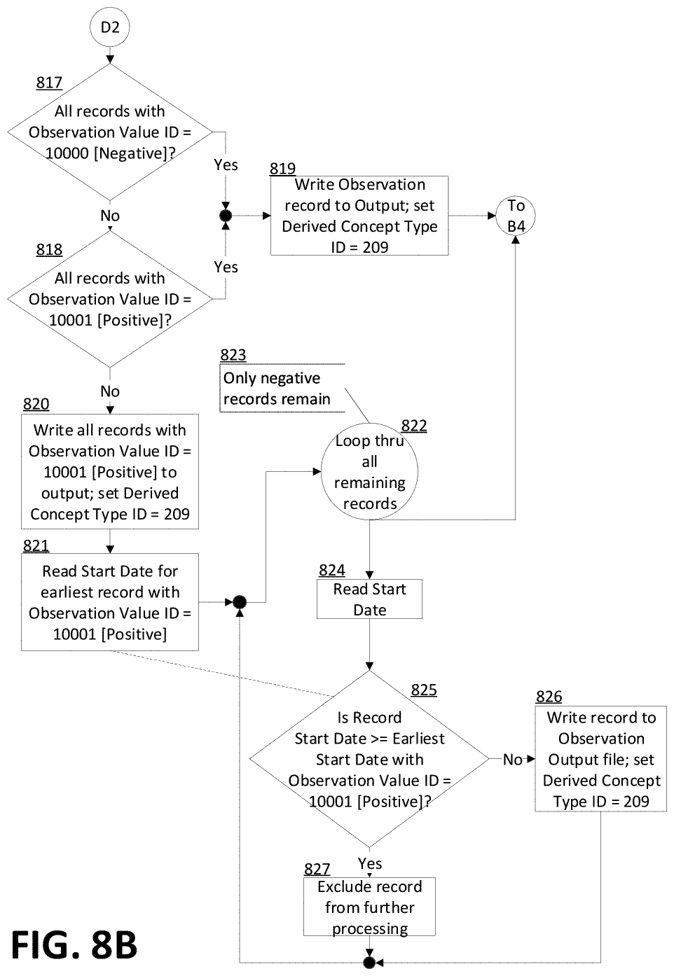

For those records remaining after execution of rules and filters of the steps of FIGS. 5-7B, the process continues as reflected in FIGS. 8A-8B for generating a data output of the pre-processing methodology. The output of this pre-processing methodology is provided as a model input data set to severity models as discussed in greater detail herein.

As noted, the sub-process of FIGS. 8A-8B is executed for all remaining observation data records as indicated at Block 801. The pre-processing system executing the pre-processing methodology determines whether all the remaining observation data records within the data set have a RAS biomarker mutation indicator as indicated at Block 802. If all observation data records remaining in the data set at the beginning of the data output generation sub-process of FIGS. 8A-8B are determined to have RAS biomarker mutation indicators, all of the observation data records are included within the output data set and the pre-processing methodology causes the pre-processing system to generate an additional derived indicator (including a derived biomarker mutation indicator and a derived result indicator) for each observation data record, and to populate the derived biomarker mutation indicator field with a RAS biomarker mutation indicator, as reflected at Block 803.

If the pre-processing system determines that the observation data records remaining for output do not all contain RAS biomarker mutation indicators, then the pre-processing methodology determines whether all of the remaining observation data records for inclusion in output include only NRAS biomarker mutation indicators and/or KRAS biomarker mutation indicators within respective biomarker mutation identifier fields, as reflected in Block 804 (with reference to a lookup table indicative of the KRAS and NRAS biomarker mutation indicators, shown at Block 805). If the pre-processing methodology determines that the remaining data records do not all contain KRAS biomarker mutation indicators and/or NRAS biomarker mutation indicators (indicating the remaining output data set includes a combination of RAS biomarker mutation indicators and KRAS biomarker mutation indicators and/or NRAS biomarker mutation indicators), the process proceeds to further analysis in accordance with FIG. 8B.

However, if all the observation data records are indicated as including only KRAS biomarker mutation indicators and/or NRAS biomarker mutation indicators, the pre-processing methodology determines whether all of the remaining observation data records have a positive result indicator (i.e., KRAS positive or NRAS positive) as reflected at Block 806. Upon determining that all of the remaining data records have either a KRAS positive result indicator or NRAS positive result indicator, all of the observation data records are included within the output data set, and the pre-processing methodology generates an additional derived indicator field for each observation data record, and populates the derived indicator field with a RAS biomarker mutation indicator, as reflected at Block 807. Accordingly, subsequent analytics, including but not limited to computer-based modeling such as machine-learning based severity models, need not distinguish between RAS, KRAS, and NRAS when training models and/or when executing models on new data sets, as these models may utilize data within the derived indicator field for training and executing models.

If the pre-processing methodology causes the pre-processing system to determine at Block 806 that not all of the observation data records are indicated as including positive result indicators, the pre-processing methodology next determines whether all of the observation data records are indicated as including negative result indicators (i.e., KRAS negative or NRAS negative) as reflected at Block 808. Upon determining that all of the remaining data records have either a KRAS negative or NRAS negative result indicator, all of the observation data records are included within the output data set, and the pre-processing system generates an additional derived biomarker mutation indicator field for each observation data record, and populates the derived biomarker mutation identifier field with a RAS biomarker mutation indicator, as reflected at Block 807 and as mentioned above.

If, according to the pre-processing methodology, the pre-processing system determines through execution of the sub-processes of Blocks 806 and 808 that the observation data records within the data set reflect a combination of positive and negative results for KRAS and/or NRAS biomarker mutation indicators, the pre-processing methodology identifies the start date for the earliest positive result indicator, as indicated at Block 809, and the pre-processing methodology includes all observation data records determined to have a positive result indicator to the output data set, generates an additional derived biomarker mutation indicator field for each observation data record, and populates the derived biomarker mutation identifier field with a RAS biomarker mutation indicator, and populates the derived biomarker mutation indicator field with a RAS biomarker mutation indicator, as reflected at Block 810.

For the remaining observation data records (those not included within the output data set in accordance with Block 810), the sub-process continues with Blocks 811-816. As indicated in the note-box of Block 812, only KRAS negative and/or NRAS negative records remain once the pre-processing methodology reaches the analysis of Block 811. As shown at Block 813, the start date for each observation data record is read for consideration, and as shown at Block 814, the pre-processing methodology determines whether the start date identified in accordance with Block 813 is on or after the start date of the earliest observation data record having a positive result indicator (as identified in Block 809). A determination that the start date of a KRAS negative result indicator or NRAS negative result indicator is on or after the earliest positive result indicator would preclude accurate assignment of a derived RAS biomarker mutation indicator, so when this occurs the record under consideration in the analysis of Blocks 811-816 is excluded from further processing. However, if the record under consideration in the analysis of Blocks 811-816 has a start date occurring before the start date of the earliest observation data record having a positive result indicator, as determined at Block 809, then the observation data record is included within the output data set, and the pre-processing methodology generates an additional derived indicator field for the observation data record and populates the derived indicator field with a RAS biomarker mutation indicator, as reflected at Block 816.

The sub-process continues as reflected in FIG. 8B with block 817. First the pre-processing methodology causes the pre-processing system to determine whether all of the observation data records that remain under consideration have a negative result indicator within a respective result indicator field, as reflected at Block 817 or a positive result indicator within a respective result indicator field, as reflected at Block 818. Note that once consideration according to Block 817 begins, it has been determined that the observation data records remaining under consideration encompass a mixture of observation data records having a RAS biomarker mutation indicator and a KRAS biomarker mutation indicator and/or NRAS biomarker mutation indicator. If all of the observation data records under consideration have a negative result indicator, or if all of the observation data records have a positive result indicator, then all of the observation data records are included within the output data set, the pre-processing methodology generates an additional derived indicator encompassing a derived biomarker mutation indicator field and a derived result indicator field and populates the derived biomarker mutation indicator field with a RAS biomarker mutation indicator, as reflected at Block 819.

Upon determining that the observation data records under consideration include a mixture of both positive results and negative results, the pre-processing methodology proceeds in accordance with the sub-process reflected at Block 820 by causing the pre-processing system to include all observation data records having a positive result indicator in the output data set. The pre-processing methodology causes the pre-processing system to generate an additional derived biomarker mutation indicator field for each observation data record, and populate the derived biomarker mutation identifier field with a RAS biomarker mutation indicator, as reflected at Block 820. The pre-processing methodology then causes the pre-processing system to identify the start date of the earliest observation data record having a positive result indicator, as indicated at Block 821, and the pre-processing system then continues by reviewing all remaining records (as indicated at Block 822). As reflected in the note of Block 823, all of the remaining observation data records still under consideration have negative result indicators.

For each of the remaining observation data records, the pre-processing methodology causes the pre-processing system to identify the start date for the observation data record, as indicated at Block 824. The pre-processing system then compares the start date retrieved at Block 824 for each observation data record having a negative result indicator with the start date of the earliest observation data record having a positive result indicator, as identified at Block 821. If, according to the date comparison reflected at Block 825, the analyzed observation data record having a negative result indicator has a start date before the start date of the earliest observation data record having a positive result indicator, as reflected in Block 821, the observation data record having the negative result indicator is included within the output data set. The pre-processing methodology causes the pre-processing system to then generate an additional derived biomarker mutation indicator field for each observation data record and populate the derived biomarker mutation identifier field with a RAS biomarker mutation indicator, as reflected at Block 826. However, determining that the observation data record having the negative result indicator has a start date on or after the start date of the earliest observation data record having a positive result indicator, the pre-processing system excludes the observation data record with the negative result indicator from further analysis (as indicated at Block 827). In this situation a negative result indicator occurring on or after a positive result indicator precludes accurate assignment of a derived RAS biomarker mutation indicator, therefore the pre-processing methodology excludes the later-generated observation data record with the negative result indicator.

### Data Output

The pre-processing process discussed above generates an output data set that is free from internal data inconsistences between included observation data records, such as inconsistencies between observation data records generated on a single date (intra-date conflicts) and/or inconsistencies between observation data records generated over a multi-day time period (inter-date conflicts) (e.g., one month, one year, and/or the like). The output data set may be provided as a model input data set to be provided to an analytic, such as a machine-learning based severity model.

As discussed above, the output data set includes a plurality of observation data records. Each of the observation data records comprises a plurality of data elements, and each observation data record may additionally comprise one or more metadata elements associated therewith. The data elements of each observation data record comprise data elements included within the originally provided observation data records (e.g., received from a data source) as well as derived data elements, such as a derived biomarker mutation indicator as discussed. In certain embodiments, the model input data set generated as output of the pre-processing system is embodied as a flat file, such that each included observation data record need not be separately accessed. Such a configuration decreases latency associated with accessing each observation data record as a separate data file. In other embodiments, the model input data set may be generated as a set of discrete observation data records.

Each observation data record may comprise data elements indicative of a first start date associated with the record, a data source identifier, concept type data (e.g., comprising an identifier indicating the record provides KRAS, NRAS, or RAS data, or the like, as provided as input for the observation data record), derived concept type data (e.g., comprising a derived identifier indicating the record provides RAS mutation data, as generated by the pre-processing system and methodology, which may differ from the concept type data), a concept condition type (cancer type) indicator, a result indicator (e.g., indicative of a positive or negative biomarker mutation indicator, as provided as input for the observation data record), a derived result indicator (e.g., indicative of a positive or negative biomarker mutation, as generated by the pre-processing system and methodology), and/or the like. In certain embodiments, one or more of the concept type data, the derived concept type data, the concept condition type indicator, the result indicator, and/or the derived result indicator may be a proprietary value selected from a corresponding ontology.

As mentioned, each observation data record of the data output may be associated with metadata providing additional data regarding the observation data record. The metadata may comprise a unique patient identifier such that the data record may be correlated with a particular patient, a unique record identifier such that an individual data record may be separately identified, a data source identifier, a log code (which may indicate a reason why the corresponding data record was excluded from the output, if applicable).

In certain embodiments, the data output may be subject to one or more limitations that are executed in accordance with various tie breaker rules. For example, the data output may be subject to a limitation that only a single output observation data record may be included within an output data set for a given start date. As mentioned above, the tiebreaker rules may be configured to eliminate duplicative observation data records for a given start date, such as by retaining an observation data record having a lower unique record identifier (selected between duplicative observation data records). Other tiebreaker rules may implement a preferred hierarchy of data sources, such that observation data records received from a more highly preferred data source (e.g., which may be indicated based on a lookup table storing the hierarchy of preferred data sources) are retained over duplicative data records received from lower ranked data sources. In such embodiments, the observation data records received from the lower ranked data sources are excluded from further analysis. Data records which are excluded from further analysis may be stored within a log data file for later auditing.

### Severity Modeling

The output data set (further referred to as a model input data set) from the output of the pre-processing methodologies, is provided as a model input data set as mentioned above. The model input data set containing clinically consistent data records for a particular cancer type having clinically consistent indication(s) of the RAS mutation status is provided to a relevant severity model that may be utilized for generating severity data indicative of attributes of the patient's colon or rectal cancer, thereby providing a high level of granularity regarding the expected costs associated with providing treatment for the patient. The data generated via the severity modeling (e.g., severity attributes) may additionally reflect other aspects of a patient's health beyond the patient's cancer, which may have an impact on the complexity (and costs) associated with treating the patient. For example, the severity models may generate severity data based at least in part on data indicative of the patient's age and gender, data indicative of comorbidities of the patient (e.g., encompassing other medical conditions that may complicate treatment of a particular cancer), and additional data indicative of the patient's condition status as relates to their cancer diagnosis. In certain embodiments, the generated severity data identifies one or more severity attributes that contributes to the severity score, such as a listing of comorbidities of the patient, one or more indications of detected RAS/KRAS/NRAS mutations, one or more markers indicative of the stage of the patient's cancer, and/or the like.

The severity models may be implemented by the management computing entity 10, and/or another computing entity. The severity models may be configured to generate a severity score that may be indicative of the relative expected treatment cost of a patient's cancer. The severity score may have no associated units (such that the severity score is simply a number that can be compared against other generated severity scores). In other embodiments, the severity score may have an associated unit, such as a cost that may be reflective of a predicted treatment cost associated with treating the patient's cancer. For example, a severity score may be reflective of a predicted medically necessary treatment cost for a patient's cancer, considering the specific circumstances of the particular patient's condition. It should be understood that other units may be utilized in certain embodiments.

The severity models may be embodied as machine-learning based models, such as regularized linear regression models (e.g., a Least Absolute Shrinkage and Selection Operator (LASSO) linear regression model), logistic regression models, neural networks, random forest models, clustering models, and/or the like, that may utilize a training data set to self-develop models for generating appropriate severity models for various cancer types. It should be understood that a single severity model may be utilized for both colon cancer and rectal cancer, or a plurality of severity models may be implemented, with each severity model corresponding to a single cancer type or other subset of model input data sets.

In certain embodiments, a training data set may be generated utilizing retrospective model input data sets generated via the above-mentioned pre-processing configurations, combined with additional data indicative of the patient's overall condition as well as data indicative of corresponding costs, that resulted from treatment of the patient's cancer. Thus, the training data sets include data reflecting inputs to the severity models as well as data reflecting an actual result corresponding with treatment of the patient's cancer—the actual result may be utilized as the dependent variable when training the model. Accordingly, the training data set may be provided for unsupervised machine learning of the severity model, thereby enabling automated generation of severity models for use in analyzing clean data sets resulting from the pre-processing configurations discussed above.

### CONCLUSION

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A computer-implemented method for automatically modeling severity attributes of a colon cancer or rectal cancer treatment utilizing a plurality of independently generated observation data records for a patient, the method comprising:
receiving a plurality of independently generated observation data records each comprising a biomarker mutation indicator relating to RAS, KRAS, or NRAS and wherein KRAS and NRAS are subsets of RAS;
generating a model input data set comprising a subset of the plurality of independently generated observation data records at least in part by:
applying a plurality of biomarker mutation indicator-based filters comprising:
an intra-date filter configured to identify a plurality of observation data records having a shared date-of-service and to eliminate one or more observation data records failing to satisfy the intra-date filter; and
an inter-date filter configured to identify a plurality of observation data records across a plurality of dates of service and to eliminate one or more observation data records failing to satisfy the inter-date filter;
wherein each of the plurality of biomarker mutation indicator-based filters are configured to identify observation data records failing to satisfy the biomarker mutation indicator-based filters across RAS, KRAS, and NRAS biomarker mutation indicators;
after applying the plurality of biomarker mutation indicator-based filters to identify the subset of the plurality of independently generated observation data records, generating a derived biomarker mutation indicator for each of the subset of the plurality of independently generated observation data records, wherein the derived biomarker mutation indicator is a derived RAS biomarker mutation indicator generated based at least in part on the biomarker mutation indicator included within a respective observation data record;
providing the model input data set to a machine-learning severity model configured to generate severity data based at least in part on the derived RAS biomarker mutation indicators of the observation data records within the model input data set; and
generating, via the machine-learning severity model, severity data relating to the patient.

2. The computer-implemented method of claim 1, wherein generating a model input data set comprises generating a flat model input data file comprising each of the subset of the plurality of observation data records.

3. The computer-implemented method of claim 1, wherein the intra-date filter is configured to eliminate one or more observation data records failing to satisfy at least one intra-date filter configuration selected from:
(A) the intra-date filter is configured to identify a first observation data record having the shared date-of-service and having a positive result indicator of at least one of RAS, KRAS, or NRAS and a second observation data record having the shared date-of-service and having a negative result indicator of at least one of RAS, KRAS, or NRAS and to eliminate the first observation data record and the second observation data record as failing to satisfy the intra-date filter;
(B) the intra-date filter is configured to identify a first observation data record having the shared date-of-service and having a positive result indicator of RAS, a second observation data record having the shared date-of-service and having a negative result indicator of KRAS, and a third observation data record having the shared date-of-service and having a negative result indicator of NRAS and to eliminate the first observation data record, the second observation data record, and the third observation data record as failing to satisfy the intra-date filter; or
(C) the intra-date filter is configured to identify a first clinical record having the shared date-of-service and having a negative result indicator of RAS and a second observation data record having the shared-date-of-service and having a positive result indicator of at least one of KRAS or NRAS and to eliminate the first observation data record and the second observation data record as failing to satisfy the intra-date filter.

4. The computer-implemented method of claim 1, wherein the inter-date filter is configured to eliminate one or more observation data records failing to satisfy at least one inter-date filter configuration selected from:
(A) the inter-date filter is configured to identify a first observation data record having a first date-of-service and having a positive result indicator of at least one RAS, KRAS, or NRAS and a second observation data record having a second date-of-service occurring after the first date-of-service and having a negative result indicator of at least one of RAS, KRAS, or NRAS and to eliminate the first observation data record and the second observation data record as failing to satisfy the inter-date filter;
(B) the inter-date filter is configured to identify a first observation data record having a first date-of-service having a positive result indicator of at least one NRAS or KRAS and a second observation data record having a second date-of-service occurring after the first-date-of-service and having a negative result indicator of RAS and to eliminate the first observation data record and the second observation data record as failing to satisfy the inter-date filter; or
(C) the inter-date filter is configured to identify a first observation data record having a first date-of-service and having a positive result indicator of at least one of RAS, KRAS, or NRAS and having a second observation data record having a second date-of-service occurring after the first date-of-service and having a negative result indicator of at least one of KRAS or NRAS and to eliminate the second observation data record as failing to satisfy the output filter.

5. The computer-implemented method of claim 1, wherein generating a model input data set is performed in accordance with a relevant data pre-processing methodology relating to colon cancer or rectal cancer, and wherein the method further comprises retrieving the relevant data pre-processing methodology from a plurality of data pre-processing methodologies based at least in part on the plurality of independently generated observation data records prior to generating the model input data set.

6. The computer-implemented method of claim 1, wherein:
the intra-date filter is configured to eliminate one or more observation data records failing to satisfy the intra-date filter relating to one of RAS, KRAS, or NRAS; and
the inter-date filter is configured to eliminate one or more observation data records failing to satisfy the inter-date filter relating to one of RAS, KRAS, or NRAS.

7. The computer-implemented method of claim 1, further comprising applying a preliminary filter criteria before generating the model input data set, wherein the preliminary filter criteria comprise one or more of:
a date-based filter criterion for selecting independently generated observation data records for further analysis as generated within a defined date range;
a data source filter criterion for selecting independently generated observation data records for further analysis as generated by one or more defined data sources; or
a data content filter criterion for selecting independently generated observation data records for further analysis as containing an identifier selected from a plurality of available identifiers eligible for further analysis.

8. The computer-implemented method of claim 1, wherein the machine-learning severity model is a linear regression model.

9. A system comprising one or more memory storage areas and one or more processors for automatically modeling severity attributes of a colon cancer or rectal cancer treatment utilizing a plurality of independently generated observation data records for a patient, the one or more processors are collectively configured to:
receive a plurality of independently generated observation data records each comprising a biomarker mutation indicator relating to RAS, KRAS, or NRAS and wherein KRAS and NRAS are subsets of RAS;
generate a model input data set comprising a subset of the plurality of independently generated observation data records at least in part by:
apply a plurality of biomarker mutation indicator-based filters comprising:
an intra-date filter configured to identify a plurality of observation data records having a shared date-of-service and to eliminate one or more observation data records failing to satisfy the intra-date filter; and
an inter-date filter configured to identify a plurality of observation data records across a plurality of dates of service and to eliminate one or more observation data records failing to satisfy the inter-date filter;
wherein each of the plurality of biomarker mutation indicator-based filters are configured to identify observation data records failing to satisfy the biomarker mutation indicator-based filters across RAS, KRAS, and NRAS biomarker mutation indicators;
after applying the plurality of biomarker mutation indicator-based filters to identify the subset of the plurality of independently generated observation data records, generate a derived biomarker mutation indicator for each of the subset of the plurality of independently generated observation data records, wherein the derived biomarker mutation indicator is a derived RAS biomarker mutation indicator generated based at least in part on the biomarker mutation indicator included within a respective observation data record;
provide the model input data set to a machine-learning severity model configured to generate severity data based at least in part on the derived RAS biomarker mutation indicators of the observation data records within the model input data set; and
generate, via the machine-learning severity model, severity data relating to the patient.

10. The system of claim 9, wherein the intra-date filter is configured to eliminate one or more observation data records failing to satisfy at least one intra-date filter configuration selected from:
(A) the intra-date filter is configured to identify a first observation data record having the shared date-of-service and having a positive result indicator of at least one of RAS, KRAS, or NRAS and a second observation data record having a shared biomarker mutation indicator and the shared date-of-service and having a negative result indicator of at least one of RAS, KRAS, or NRAS and to eliminate the first observation data record and the second observation data record as failing to satisfy the intra-date filter;
(B) the intra-date filter is configured to identify a first observation data record having the shared date-of-service and having a positive result indicator of RAS, a second observation data record having the shared date-of-service and having a negative result indicator of KRAS, and a third observation data record having the shared date-of-service and having a negative result indicator of NRAS and to eliminate the first observation data record, the second observation data record, and the third observation data record as failing to satisfy the intra-date filter; or
(C) the intra-date filter is configured to identify a first clinical record having the shared date-of-service and having a negative result indicator of RAS and a second observation data record having the shared-date-of-service and having a positive result indicator of at least one of KRAS or NRAS and to eliminate the first observation data record and the second observation data record as failing to satisfy the intra-date filter.

11. The system of claim 9, wherein the inter-date filter is configured to eliminate one or more observation data records failing to satisfy at least one inter-date filter configuration selected from:
(A) the inter-date filter is configured to identify a first observation data record having a first date-of-service and having a positive result indicator of at least one RAS, KRAS, or NRAS and a second observation data record having a second date-of-service occurring after the first date-of-service and having a negative result indicator of at least one of RAS, KRAS, or NRAS and to eliminate the first observation data record and the second observation data record as failing to satisfy the inter-date filter;
(B) the inter-date filter is configured to identify a first observation data record having a first date-of-service having a positive result indicator of at least one NRAS or KRAS and a second observation data record having a second date-of-service occurring after the first-date-of-service and having a negative result indicator of RAS and to eliminate the first observation data record and the second observation data record as failing to satisfy the inter-date filter; or
(C) the inter-date filter is configured to identify a first observation data record having a first date-of-service and having a positive result indicator of at least one of RAS, KRAS, or NRAS and having a second observation data record having a second date-of-service occurring after the first date-of-service and having a negative result indicator of at least one of KRAS or NRAS and to eliminate the second observation data record as failing to satisfy the output filter.

12. The system of claim 9, wherein:
the intra-date filter is configured to eliminate one or more observation data records failing to satisfy the intra-date filter relating to one of RAS, KRAS, or NRAS; and
the inter-date filter is configured to eliminate one or more observation data records failing to satisfy the inter-date filter relating to one of RAS, KRAS, or NRAS.

13. The system of claim 9, wherein the one or more processors are additionally configured to apply a preliminary filter criteria before generating the model input data set, wherein the preliminary filter criteria comprise one or more of:
a date-based filter criterion for selecting independently generated observation data records for further analysis as generated within a defined date range;
a data source filter criterion for selecting independently generated observation data records for further analysis as generated by one or more defined data sources; or
a data content filter criterion for selecting independently generated observation data records for further analysis as containing an identifier selected from a plurality of available identifiers eligible for further analysis.

14. The system of claim 9, wherein the machine-learning severity model is a linear regression model.

15. A computer program product for automatically modeling severity attributes of a colon cancer or rectal cancer treatment utilizing a plurality of independently generated observation data records for a patient, the computer program product comprising at least one non-transitory computer-readable storage medium having computer-readable program code portions stored therein, the computer-readable program code portions configured to:
receive a plurality of independently generated observation data records each comprising a biomarker mutation indicator relating to RAS, KRAS, or NRAS and wherein KRAS and NRAS are subsets of RAS;
generate a model input data set comprising a subset of the plurality of independently generated observation data records at least in part by:
apply a plurality of biomarker mutation indicator-based filters comprising:
an intra-date filter configured to identify a plurality of observation data records having a shared date-of-service and to eliminate one or more observation data records failing to satisfy the intra-date filter; and
an inter-date filter configured to identify a plurality of observation data records across a plurality of dates of service and to eliminate one or more observation data records failing to satisfy the inter-date filter;
wherein each of the plurality of biomarker mutation indicator-based filters are configured to identify observation data records failing to satisfy the biomarker mutation indicator-based filters across RAS, KRAS, and NRAS biomarker mutation indicators;
after applying the plurality of biomarker mutation indicator-based filters to identify the subset of the plurality of independently generated observation data records, generate a derived biomarker mutation indicator for each of the subset of the plurality of independently generated observation data records, wherein the derived biomarker mutation indicator is a RAS biomarker mutation indicator generated based at least in part on the biomarker mutation indicator included within a respective observation data record;
provide the model input data set to a machine-learning severity model configured to generate severity data based at least in part on the derived RAS biomarker mutation indicators of the observation data records within the model input data set; and
generate, via the machine-learning severity model, severity data relating to the patient.

16. The computer program product of claim 15, wherein the intra-date filter is configured to eliminate one or more observation data records failing to satisfy at least one intra-date filter configuration selected from:
(A) the intra-date filter is configured to identify a first observation data record having the shared date-of-service and having a positive result indicator of at least one of RAS, KRAS, or NRAS and a second observation data record having the shared date-of-service and having a negative result indicator of at least one of RAS, KRAS, or NRAS and to eliminate the first observation data record and the second observation data record as failing to satisfy the intra-date filter;
(B) the intra-date filter is configured to identify a first observation data record having the shared date-of-service and having a positive result indicator of RAS, a second observation data record having the shared date-of-service and having a negative result indicator of KRAS, and a third observation data record having the shared date-of-service and having a negative result indicator of NRAS and to eliminate the first observation data record, the second observation data record, and the third observation data record as failing to satisfy the intra-date filter; or
(C) the intra-date filter is configured to identify a first clinical record having the shared date-of-service and having a negative result indicator of RAS and a second observation data record having the shared-date-of-service and having a positive result indicator of at least one of KRAS or NRAS and to eliminate the first observation data record and the second observation data record as failing to satisfy the intra-date filter.

17. The computer program product of claim 15, wherein the inter-date filter is configured to eliminate one or more observation data records failing to satisfy at least one inter-date filter configuration selected from:
(A) the inter-date filter is configured to identify a first observation data record having a first date-of-service and having a positive result indicator of at least one RAS, KRAS, or NRAS and a second observation data record having a second date-of-service occurring after the first date-of-service and having a negative result indicator of at least one of RAS, KRAS, or NRAS and to eliminate the first observation data record and the second observation data record as failing to satisfy the inter-date filter;
(B) the inter-date filter is configured to identify a first observation data record having a first date-of-service having a positive result indicator of at least one NRAS or KRAS and a second observation data record having a second date-of-service occurring after the first-date-of-service and having a negative result indicator of RAS and to eliminate the first observation data record and the second observation data record as failing to satisfy the inter-date filter; or
(C) the inter-date filter is configured to identify a first observation data record having a first date-of-service and having a positive result indicator of at least one of RAS, KRAS, or NRAS and having a second observation data record having a second date-of-service occurring after the first date-of-service and having a negative result indicator of at least one of KRAS or NRAS and to eliminate the second observation data record as failing to satisfy the output filter.

18. The computer program product of claim 15, wherein:
the intra-date filter is configured to eliminate one or more observation data records failing to satisfy the intra-date filter relating to one of RAS, KRAS, or NRAS; and
the inter-date filter is configured to eliminate one or more observation data records failing to satisfy the inter-date filter relating to one of RAS, KRAS, or NRAS.

19. The computer program product of claim 15, wherein the computer-readable program code portions are further configured to apply a preliminary filter criteria before generating the model input data set, wherein the preliminary filter criteria comprise one or more of:
a date-based filter criterion for selecting independently generated observation data records for further analysis as generated within a defined date range;
a data source filter criterion for selecting independently generated observation data records for further analysis as generated by one or more defined data sources; or
a data content filter criterion for selecting independently generated observation data records for further analysis as containing an identifier selected from a plurality of available identifiers eligible for further analysis.

20. The computer program product of claim 15, wherein the machine-learning severity model is a linear regression model.
